(19) United States
(12) Patent Application Publication
(10) Pub. No.: US 2002/0120404 A1
(43) Pub. Date: Aug. 29, 2002

(54) Methods and apparatus for mass fingerprinting of biomolecules

(76) Inventor: Kenneth C. Parker, Hopkinton, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/745,920

(22) Filed: Dec. 21, 2000

(51) Int. Cl. G06F 19/00; G01N 33/48; G01N 33/50

(52) U.S. Cl. 702/19

(57) ABSTRACT

Methods and apparatus that determine molecules in a sample by mass fingerprinting are disclosed. In one embodiment, the method comprises: (1) comparing the mass signals of a mass spectrum to a biomolecule fragment signal list to determine mass signal-biomolecule fragment matches; (2) quantifying the mass signal-biomolecule fragment match significance based on a biomolecule fragment detection parameter; (3) quantifying a biomolecule's likelihood of being present in the sample based on the quantified significance of mass signal-biomolecule fragment matches; and (4) determining the likelihood of the presence of biomolecule(s) in the sample by comparison of the quantified biomolecule presence likelihood. The invention also provides an apparatus for determining the likelihood of the presence of a biomolecule in a sample using the biomolecule fragment detection parameters of biomolecule fragments matched to the mass signals of a mass spectrum of the sample.

### FIELD OF THE INVENTION

The invention relates generally to the field of molecule identification. In particular, the invention relates to biomolecule identification by mass fingerprinting.

### BACKGROUND OF THE INVENTION

Mass fingerprinting is an analytical technique for determining the presence of molecules in a sample. Typically, a sample is ionized and a mass spectrum of the ionized sample molecules is obtained by mass spectrometry. A sample may contain a single constituent molecule to many hundreds or thousands of molecules. The presence of a molecule in the sample is determined by correlating the information contained in the sample mass spectrum with known or theoretical mass spectra for the molecule. Mass fingerprinting is of particular importance in the area of proteome analysis. Proteome analysis is the measurement of protein expression in a biological sample to characterize biological processes, such as disease and mechanisms of gene expression. Understanding protein expression is crucial to a complete understanding of biological systems. Unlike mRNA, which only acts as a disposable messenger, proteins implement almost all controlled biological functions and, as a result, are integral to such functions as normal cell activity, disease processes, and drug responses. However, protein expression is not predictable. First, protein expression is not predictable from mRNA expression maps because mRNA transcript levels are not strongly correlated with protein levels. Second, proteins are dynamically modified in biological systems by environmental factors in ways which are not predictable from genetic information. Accordingly, knowledge of a biological system's response to drugs or disease typically requires a comparison of many “normal” and “abnormal” samples. Thus, proteome analysis requires the determination of the proteins present in a variety of samples.

Determination of the proteins in a sample is complicated by several factors not present for other biomolecules. First, unlike DNA and RNA, proteins cannot currently be amplified. Second, it is often difficult to purify proteins from a sample for separate analysis. Third, there are few solvents in which all proteins are soluble and which are compatible with protein analysis techniques. However, the largest obstacle to an analysis of the proteins present in a sample is often the shear number that can be present. For example, a cell typically contains thousands of proteins. Accordingly, a mass spectrum of a typical sample often contains hundreds or thousands of mass signals of the proteins and their peptide fragments. Therefore, any attempt to identify a protein present in such a sample must be able to accurately correlate the appropriate mass signals from amongst the hundreds or thousands of irrelevant and “background” mass signals in the spectrum.

### SUMMARY OF THE INVENTION

The present invention provides methods and apparatus that determine the constituent molecules in a biological sample by mass fingerprinting. Biomolecules amenable to determination by the present invention include, but are not limited to, proteins, carbohydrates, and oligonucleotides. The present invention may provide increased accuracy, relative to currently available mass fingerprinting techniques, where the biological sample contains hundreds or even thousands of constituent molecules. The accuracy of the methods of the present invention is accomplished by explicitly taking into account the likelihood of detecting a biomolecule fragment as a mass signal in the mass spectrum of the sample. The numerical value associated with the likelihood of detecting a biomolecule fragment from a given biomolecule is referred to as a “biomolecule fragment detection parameter.” According to the present invention, the biomolecule fragment detection parameter(s) is used to quantify the significance of a correlation of a mass signal in the mass spectrum with a biomolecule fragment.

The increased accuracy afforded by the present invention provides several advantages. For example, it improves the determination of: (1) the identity of the constituent(s) present in a sample (2) the amount of identified constituent(s) present in a sample; and (3) the approximate abundance of any unidentified constituent. Moreover, the reliability of the above determinations according to the present invention are less sensitive to: (1) the number of mass signals employed in the correlation with known or theoretical mass spectra of a molecule; and (2) the mass tolerance limit used for ascertaining matches between measured mass signals and a theoretical mass spectrum for a molecule. Accordingly, the methods of the present invention enable the investigator to compare a large number of mass signals. Further, the present invention improves the efficiency of mass fingerprinting by using a greater portion of the sample mass spectrum's information content than current mass fingerprinting techniques. Accordingly, the present invention may provide increased accuracy in multiple molecule identifications and in biomolecule identifications that employ less common methodologies, such as the use of less commonly studied enzymes to digest a sample's proteins.

In one aspect, the present invention features a method of determining the biomolecules present in a biological sample based on a mass spectrum of the sample. A mass spectrum is composed of at least one mass signal, however, a spectrum is typically composed of more than a hundred mass signals. Each mass signal has an intensity and an associated mass. Herein, the term “signal intensity” is meant to refer to the intensity associated with a mass signal regardless of whether the intensity is an absolute signal intensity, a corrected signal intensity, a relative signal intensity, or a signal-to-noise parameter. Herein, the term “signal mass” is meant to refer to the mass associated with a mass signal.

In one embodiment, a sample containing biomolecules is subjected to a fragmentation process, such as a digest, that yields fragments of the sample's constituent biomolecules. In this embodiment, a mass spectrum of the resulting biomolecule pieces is obtained that consists of a set of mass signals which primarily correspond to the biomolecule fragments that make up the biomolecules present in the sample. In one embodiment, the biomolecules comprise proteins; and, accordingly, the biomolecule fragments comprise peptides. The mass signals are compared to a biomolecule fragment signal list that contains the biomolecule fragments known or predicted to be generated by the digestion and/or fragmentation of a given biomolecule. That is, the list may contain biomolecule fragment signals experimentally known to be generated and/or theoretically predicted to be generated for an observed or predicted biomolecule or in other words, “knowable” biomolecule fragment signals. The list of known or predicted biomolecule fragments may comprise one or more databases and/or be determined as desired. For example, the list may be determined by on-the-fly calculation or on-line determination. The list may contain biomolecule fragment signals of not only whole biomolecules but also fragment signals of contiguous segments of a biomolecule. In addition, the list may contain biomolecule fragment masses that have been adjusted to account for adducts or modifications. Accordingly, when reference is made to a list of “knowable” biomolecule fragment signals, it is to be understood to mean a list of biomolecule fragments known and/or predicted to be generated by the digestion and/or fragmentation of a given biomolecule(s) or biomolecule segment(s).

In the comparison of a mass signal to a list of knowable biomolecule fragment signals, the signal mass is compared to the mass of the biomolecule fragment in the list. If the signal mass is equal to the mass of the biomolecule fragment, within a selected error range, the mass signal is considered to correspond to the biomolecule fragment or, in other words, the mass signal is said to “match” the biomolecule fragment. Accordingly, when reference is made to “matching” herein, it is to be understood to mean a comparison of two numerical values which will be considered “matched” if they are equal within an error range. For example, the value “1” is considered equal to and matched to the value “2” if the error range is greater than or equal to plus or minus 1. When a signal mass matches a biomolecule fragment mass, the biomolecule or biomolecules in the list associated with that biomolecule fragment mass are considered as potential source(s) for the corresponding mass signal of the matched signal mass. Such a biomolecule is referred to herein as a “potential source biomolecule.”

According to the methods of the invention, a numerical value is then calculated and assigned to the potential source biomolecule; this numerical value is referred to as the “biomolecule score.” It should be realized that the present invention provides several formulae for determining biomolecule scores. Regardless, the biomolecule score explicitly takes into account the likelihood of detecting the biomolecule fragment(s) of the potential source biomolecule that match signal mass(es) of the mass spectrum. The biomolecule score is a measure of the probability that the potential source biomolecule is indeed the source of a mass signal in the mass spectrum and, accordingly, is a measure of the likelihood that the biomolecule is present in or absent from the sample. The likelihood of the presence or absence of a biomolecule is determined by comparing the biomolecule scores of the potential source biomolecules. In one embodiment, the potential source biomolecule with the biomolecule score corresponding to the highest presence likelihood is considered present in the sample.

In one embodiment, the biomolecule(s) of interest comprise carbohydrates. Examples of such carbohydrates include, but are not limited to, cellulose, starches, pectins, saccharides, sugar phosphates, and glycosides. The carbohydrates of interest may be monomeric or polymeric. For example, a polymeric glucose such as glycogen may be of interest. The carbohydrates can be obtained from a complex mixture of carbohydrates or, for example, from proteins or cells by chemical or enzymatic release. The carbohydrates may be separated by high pressure liquid chromatography (HPLC) and the resulting eluents subjected to matrix assisted laser desorption ionization time-of-flight (MALDI-TOF) analysis. For example, in one embodiment the carbohydrates of interest comprise polysaccharides. The carbohydrates in an eluent are subjected to a fragmentation process, such as an enzyme digest, a Ruff degradation, a Wohl degradation, etc. Suitable enzyme digests include, but are not limited to, starch digesting enzymes such as α-amylases and β-amylases. In this embodiment, the biomolecule fragments comprise sugar subunits of the polysaccharides. The resulting mass spectrum of an eluent is then compared, using the methods of the present invention, to a list of fragments known or predicted to be generated by the fragmentation process employed.

In another embodiment, the invention features a method to determine which mass signals from a sample's mass spectrum should be analyzed by MS-MS to confirm or reject determinations of biomolecules and/or biomolecule fragments as likely present in or absent from the sample. In this embodiment, a mass spectrum of the resulting biomolecule fragments is obtained that produces a first spectrum of mass signals which primarily correspond to the biomolecule fragments that make up the biomolecules present in the sample. The signal masses are compared to biomolecule fragment masses in a biomolecule fragment signal list that contains the biomolecule fragments known or predicted to be generated by the digestion and/or fragmentation of a given biomolecule. When a signal mass matches a biomolecule fragment mass, any biomolecule associated with that biomolecule fragment in the list is considered a potential source biomolecule for the corresponding mass signal. A biomolecule score is then assigned to the potential source biomolecule(s). The biomolecule fragment scores and/or biomolecule scores are then used to determine whether a mass signal in the first mass spectrum is subjected to further mass analysis. In one embodiment, only mass signals corresponding to a potential source biomolecule with a high likelihood of being present, as indicated by the biomolecule score, are subjected to MS-MS analysis to confirm or reject the identification and the likelihood of the presence or absence of the biomolecule in the sample. In another embodiment, mass signals that correspond only to a potential source biomolecule with a very low likelihood of being present, or which correspond to no biomolecule in the list, are subject to MS-MS analysis. In another embodiment, mass signal(s) that correspond to two or more potential source biomolecules with comparable biomolecule scores are subjected to MS-MS analysis to confirm or reject the source biomolecule(s) of the mass signals.

In another aspect, the present invention provides an apparatus for determining the presence of a biomolecule in a biological sample using the biomolecule fragment detection parameter(s). In one embodiment, the apparatus includes: (1) a mass spectrometry instrument for obtaining a mass spectrum of a sample; (2) memory elements for storing the mass spectrum, mass signal-biomolecule fragment matches, biomolecule fragment scores and biomolecule scores determined according to the methods of the present invention; (3) a memory element for storing a list of biomolecule fragments known or predicted to be generated by the digestion and/or fragmentation of select biomolecules; (4) a memory element containing a comparator that compares mass signals to the database to determine whether a mass signal matches a biomolecule fragment in a database; (5) a memory element containing a weight generator that determines a numerical value referred to as a biomolecule fragment score that quantifies the significance of a mass signal-biomolecule fragment match; (6) a memory element containing a combination generator that combines the biomolecule fragment scores of an associated select biomolecule to determine a biomolecule score for the biomolecule; and (7) an output device that enables an investigator to compare biomolecule scores and thereby determine the likelihood of the presence or absence of a biomolecule in the biological sample. In a preferred embodiment, the mass spectrometer instrument is a matrix assisted laser desorption ionization time-of-flight (“MALDI-TOF”) instrument. In a preferred embodiment, the memory elements are portions of the random access memory of a computer.

In another aspect, the present invention provides an article of manufacture where the functionality of the method of the present invention is embedded on a computer-readable program means, such as, but not limited to, a floppy disk, a hard disk, an optical disk, a magnetic tape, a PROM, an EPROM, or CD-ROM.

The foregoing, and other features and advantages of the invention, as well as the invention itself, will be more fully understood from the description, drawings, and claims which follow. Although mass fingerprinting of a biomolecule is often described in the context of specific biomolecules, such as proteins, it is noted that other biomolecules can be mass fingerprinted without departing from the spirit and scope of the invention.

### BRIEF DESCRIPTION OF THE DRAWINGS

FIG. 1 is a flow diagram illustrating an embodiment of a mass fingerprinting method according to the present invention.

FIG. 2 is a flow diagram showing various embodiments which may be included in step 1000 in FIG. 1.

FIG. 3 is a flow diagram showing various embodiments which may be included in step 2000 in FIG. 1.

FIG. 4A is a flow diagram showing a detail of an embodiment which may be included in step 3000 in FIG. 1.

FIGS. 4B-E are flow diagrams showing various embodiments of the steps of FIG. 4A.

FIG. 5 is a flow diagram showing various embodiments which may be included in step 4000 in FIG. 1.

FIG. 6 is a flow diagram showing various embodiments which may be included in step 5000 in FIG. 1.

FIG. 7 is a diagrammatic representation illustrating an embodiment of an apparatus for practicing the method of the present invention.

### DETAILED DESCRIPTION

Samples appropriate and amenable to the methods and apparatus of the present invention include, but are not limited to, tissue extracts, chromatographic fractions, slices from SDS gels, or spots from two-dimensional gels. In one embodiment, the sample is digested, preferably with an enzyme. Suitable enzymes include, but are not limited to, carboxypeptidasees, aminopeptidases, trypsin, and pepsin, or if the sample contained nucleic acids, exonucleases, and endonucleases, including restriction endonucleases. In one embodiment, cyanogen bromide, which cleaves biomolecule sequences after the amino acid methionine, is used in place of an enzyme. In another embodiment, the enzyme is trypsin. It is also preferred that small molecule contaminants are removed from the sample prior to acquisition of the mass spectrum. Suitable contaminant removal steps include, but are not limited to, reversed-phase chromatography and/or extensive washing of biomolecule slices or spots from polyacrylamide gels.

Any suitable mass spectrometry technique can be used to obtain a mass spectrum of the sample under investigation. Suitable mass spectrometry techniques include any suitable sample ionization technique coupled with any suitable mass spectrometer. Suitable ionization techniques include, but are not limited to, matrix assisted laser desorption ionization (MALDI), and electrospray ionization. Suitable mass spectrometers include, but are not limited to, time-of-flight (TOF) instruments, magnetic sector instruments, ion traps, reflectrons, Fourier transform instruments, guide wire instruments, and radio-frequency instruments such as quadrupoles and other multi-pole instruments. In addition, suitable mass spectrometry techniques further include any technique that can separate biomolecules or biomolecule fragments such as, for example, one-dimensional gel electrophoresis, two-dimensional gel electrophoresis, capillary electrophoresis, gas phase chromatography (GPC), and high pressure liquid chromatography (HPLC). In a preferred embodiment the mass spectrometry technique is MALDI-TOF.

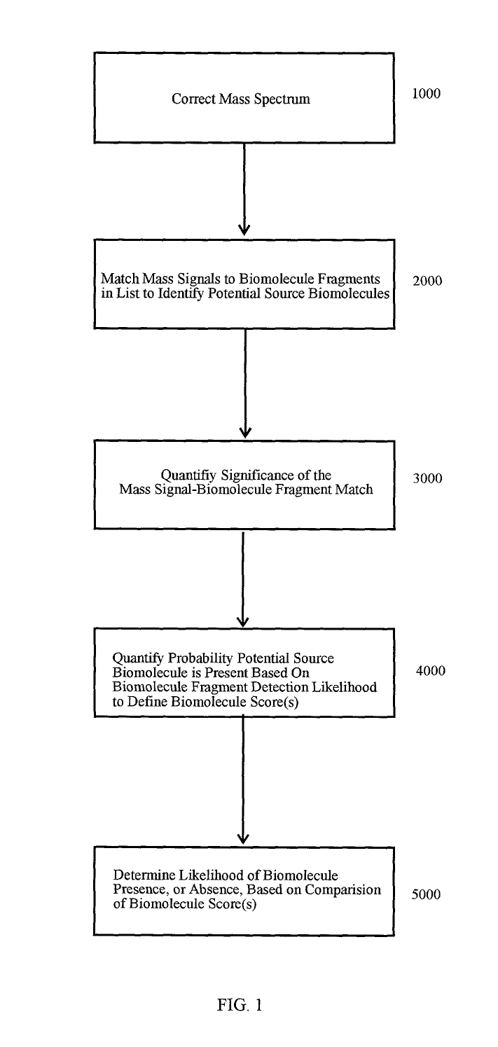

Referring to FIG. 1, an embodiment of a method according to the present invention for analyzing a mass spectrum of a sample to determine the likelihood of the presence or absence of a biomolecule in the sample is shown. In one embodiment, the method performs five general steps after a mass spectrum of a sample is provided or obtained directly: (1) correcting the mass spectrum 1000; (2) matching mass signals to biomolecule fragments and identifying potential source biomolecules for the mass signals 2000; (3) quantifying the mass signal-biomolecule fragment match significance 3000; (4) quantifying the biomolecule presence likelihood 4000; and (5) determining the likelihood that the biomolecule(s) is present or absent based on the quantified presence likelihood 5000. Each of these steps will be generally described below, followed by a more detailed discussion.

Step 1000 adjusts the calibration of the mass scale of the mass spectrum. In addition, step 1000 corrects for, for example, noise, spurious signals, random errors or systemic errors arising from the mass spectrometry technique used to obtain the mass spectrum. Such correction includes, but is not limited to, data smoothing, Fourier Transform filtration, baseline subtraction, and convolution with an instrument response function. In addition, step 1000 corrects for, for example, mass signals not of interest or isotopic variation in mass signals of interest. Such correction, includes, but is not limited to, signal subtraction, signal conflation, and signal attenuation. However, the precise extent and degree of any correction in step 1000 is determined based upon, for example, the probability that noise, errors, contaminant signals, and the like, might interfere with the determination of a biomolecule's absence or presence likelihood, and/or upon investigator convenience. Further, it is not central to the present invention whether the mass spectrum is corrected, for example, certain embodiments may not require correction.

Step 2000 compares at least one selected mass signal of the mass spectrum to a knowable list of biomolecule fragments to determine if the corresponding signal mass matches a biomolecule fragment mass known or predicted to be generated by the digestion and/or fragmentation of a given biomolecule. It should be realized that the listed biomolecule fragment mass may be corrected for adducts or modifications before comparison to a signal mass. Such adducts and modifications can arise, for example, from the sample preparation, digestion, and/or fragmentation techniques employed. The signal mass is compared to at least one biomolecule fragment of at least one biomolecule in the list. The step of comparing the signal mass to biomolecule fragments is then repeated for each biomolecule fragment of the selected biomolecule in the list. It is to be realized that more than one mass signal may match a given biomolecule fragment of a given biomolecule. Step 2000 is reiterated for other selected mass signals until all selected mass signals have been compared to biomolecule fragments in the list. The precise number of mass signals that are to be compared, and the biomolecule fragments of which biomolecules they are to be compared to, is determined based upon various factors such as the biomolecule to be detected, the nature of the sample, the mass range of the mass spectrum and/or investigator convenience.

In another embodiment, mass signals are also compared 2000 to the fragments of contiguous segments, i.e., sub-portions, of biomolecules in the list. The “length” of the segments is defined by a selected segment molecular weight limit. Accordingly, only mass signal-biomolecule fragment matches that correspond to biomolecule fragments that all appear in the selected “length” of a biomolecule segment are used to calculate a biomolecule score for that biomolecule segment. For example, where the biomolecules of interest comprise proteins, mass signals are also compared to the peptides, i.e., biomolecule fragments, of contiguous segments of proteins, that is, protein fragments, of a selected molecular weight. The steps of matching 2000, quantifying match significance 3000, and quantifying the likelihood that the biomolecule segment is present 4000, are all performed as for a whole biomolecule. In one embodiment, a biomolecule segment molecular weight limit of 50,000 Daltons is preferred, if, for example, the biomolecule of interest is known or has been found to have a molecular weight of about 50,000 Daltons.

Referring again to step 2000, if a signal mass matches a biomolecule fragment mass, the biomolecule associated with that biomolecule fragment in the list may be a potential source for that corresponding mass signal. Accordingly, it is to be realized that multiple biomolecules may be considered as potential source biomolecules for a single mass signal. In one embodiment, a single match is sufficient for a biomolecule to be considered a potential source biomolecule for the matched mass signal. In another embodiment, a minimum number of mass signals must match the biomolecule fragments of a biomolecule before the associated biomolecule is considered a potential source biomolecule for those matched mass signals. In another embodiment, the mass signal-biomolecule fragment matches must satisfy other criteria in addition to the number of matches before the biomolecule associated with the matched biomolecule fragment is considered a potential source biomolecule. For example, each matched mass signal may be required to have a minimum signal intensity and/or each matched biomolecule fragment may be required to have a minimum likelihood of detection before the associated biomolecule is considered a potential source biomolecule.

Step 3000 quantifies the significance of a match of a biomolecule fragment to a signal mass in step 2000. Specifically, a numerical value is determined and assigned to a selected matched biomolecule fragment that is a measure of the significance of the match for identifying a potential source biomolecule. This numerical value is referred to as a “biomolecule fragment score.” Step 3000 is then reiterated for other selected biomolecule fragment masses until all selected biomolecule fragments of all selected potential source biomolecules have been assigned a biomolecule fragment score. The precise number of biomolecule fragment masses and potential source biomolecules that are selected is determined based upon, for example, factors such as the biomolecule to be detected, the complexity of the mass spectrum, the mass range of the mass spectrum and/or investigator convenience. The biomolecule fragment score is a function of the biomolecule fragment detection parameter; i.e., the biomolecule fragment score is a function of the likelihood of detecting the matched biomolecule fragment as a fragment and/or digestion product of the associated potential source biomolecule with a given digest and a given mass spectrometry technique. Accordingly, it is to be realized that a single matched biomolecule fragment may have more than one biomolecule fragment score. For instance, a biomolecule fragment can occur at multiple locations throughout a biomolecule and/or more than one mass signal may match a single biomolecule fragment, thereby producing more than one biomolecule fragment score. For example, a peptide (i.e., a bimolecule fragment) of a protein can occur at multiple locations throughout a protein (i.e., the biomolecule); as a result, the same peptide sequence occurs but with different amino acid numbers.

In one embodiment, the biomolecule fragment score is also a function of the closeness of the match as expressed by the difference between the signal mass and matched biomolecule fragment mass. In another embodiment, the biomolecule fragment score is further a function of the intensity of the corresponding matched mass signal. Step 4000 quantifies the likelihood that a potential source biomolecule identified in step 2000 is present in the sample based on the biomolecule fragment scores determined and assigned in step 3000. Specifically, a numerical value is calculated and assigned to the potential source biomolecule that is a measure of the likelihood, or probability, that the potential source biomolecule is indeed the source of a mass signal or signals in the mass spectrum. (As discussed above, this numerical value is referred to as the “biomolecule score.”) Accordingly, the biomolecule score is a measure of the likelihood that the corresponding biomolecule is present in the sample. The biomolecule score explicitly takes into account the biomolecule fragment detection parameters, i.e. detection likelihoods, of the biomolecule fragment(s) of the potential source biomolecule that matched signal mass(es) of the mass spectrum. Step 4000 is then reiterated for other selected potential source biomolecules until each has been assigned a biomolecule score. The precise number of potential source biomolecules that are selected is determined based upon the nature of the sample, the biomolecule to be detected, the complexity of the mass spectrum and/or investigator convenience, among other factors.

Step 5000 determines the likelihood that a biomolecule is present in or absent from the sample based on the biomolecule scores of the potential source biomolecules. Specifically, the biomolecule scores are evaluated based on the likelihood that the corresponding potential source biomolecule is present in the sample. In one embodiment, the higher the biomolecule score, the higher the likelihood that the potential source biomolecule is present in the sample. However, it is not central to the present invention whether an increasing, decreasing, positive or negative biomolecule score corresponds to the greater likelihood that the potential source biomolecule is present. Rather, the methods of the present invention require only that the likelihood, or probability, associated with a biomolecule score is substantially a monotonic function of the biomolecule score.

The operation of these five general steps will be explained in greater detail in connection with the following detailed description of each step, including various embodiments of the present invention. In the interest of clarity and to aid those of ordinary skill in the art in understanding the present invention, the description below often uses proteins as an example biomolecule to illustrate the principles of the present invention. Accordingly, the biomolecule fragments of the proteins correspond to and are referred to as peptides in the description below. It is to be realized that although the methods and apparatuses of the present invention may be discussed in terms of proteins and peptides, the present invention is not so limited and is applicable to biomolecules in general, such as, for example, to carbohydrates and oligonucleotides.

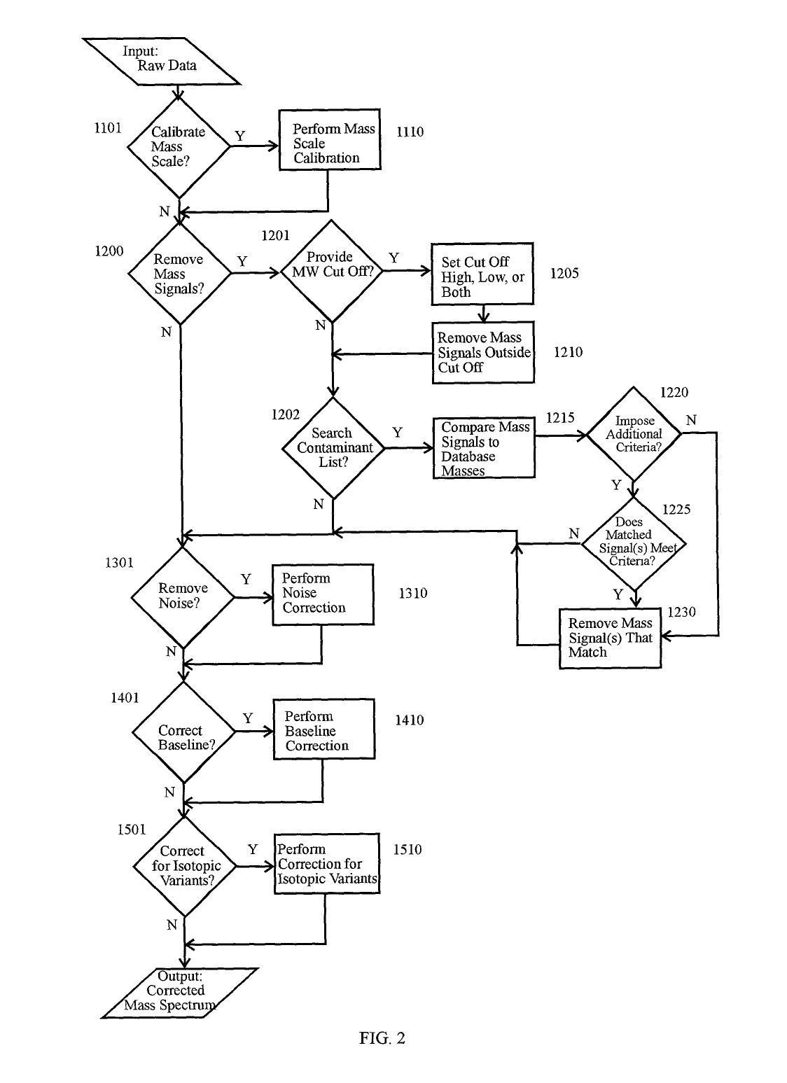

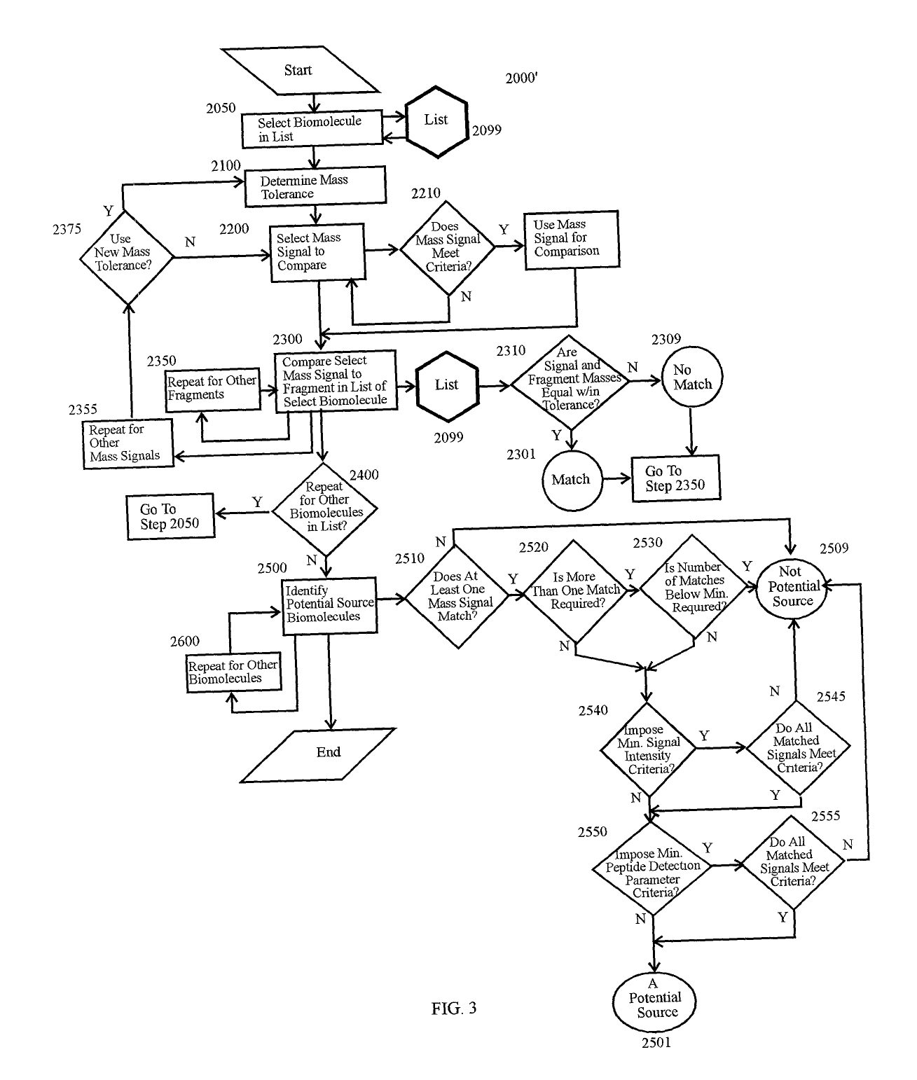

As noted above, a mass spectrum of the sample under investigation is obtained directly or provided. The mass spectrum typically is in an electronic format that includes the raw data. More often than not, it is desirable to “correct” the raw data that constitutes the mass spectrum. Referring to FIG. 2, various embodiments of corrections to a mass spectrum which may be included in general step 1000 are shown. It is to be understood that while FIG. 2 illustrates correcting the mass spectrum in a specific order, the order in which steps 1101 to 1501 are performed it is not central to the present invention and may vary, and one or more steps may be eliminated. Typically, an investigator first desires to calibrate the mass scale of the mass spectrum. If calibration of the mass scale is desired (“YES” to test 1101), calibration 1110 is accomplished by any suitable method known in the art.

Second, an investigator often wishes to remove mass signals from the mass spectrum (“YES” to test 1200). Such signals may, for example, arise from contaminants, fall outside the reliable detection range of the mass spectrometer and/or correspond to molecules not of interest. The investigator may wish to remove mass signals that fall below a lower molecular weight (“MW”) cutoff, above an upper molecular weight cutoff, or both. If an investigator decides to provide a molecular weight cutoff value (“YES” to test 1201), a lower molecular weight cutoff, an upper molecular weight cutoff, or both 1205, are selected and the mass signals which fall outside the cut off value are removed 1210 from the mass spectrum before further analysis.

In addition, in one embodiment, commonly encountered contaminants can be corrected for by searching a supplementary list containing masses associated with such contaminants (“YES” to test 1202). Signal masses are compared to the masses associated with contaminants in the supplementary list 1215. In one embodiment, those signal masses in the mass spectrum that match contaminant masses are removed 1230 from the mass spectrum. In another embodiment, signal masses in the mass spectrum that match contaminant masses are removed 1230 only if the mass signal-contaminant matches satisfy additional selected criteria (“YES” to tests 1220 and 1225). Preferably, a minimum number of mass signals with a minimum signal intensity must match before the signal masses matched to a contaminant are removed from the mass spectrum. This embodiment can quantify the intensity, or relative concentration, of a contaminant and allows the number and nature of matches to contaminant masses to determine whether a contaminant is present instead of blindly subtracting the mass signals of commonly found contaminants. This embodiment is especially powerful in cases where the contaminant in question is not actually a biomolecule of interest, but a collection of mass signals commonly encountered together. Examples of such contaminants include polymers like silicone oil and polyethylene glycol, mixtures of calibration standards, and masses that derive from the matrix in MALDI or the solvent in electrospray ionization techniques.

In another embodiment, the mass spectrum is also processed to remove electronic noise from, and correct the baseline of, the mass spectrum (“YES” to tests 1301 and 1401, respectively). Electronic noise removal 1310 and baseline correction 1410 are accomplished by any suitable method known in the art. More importantly, an investigator often desires to correct for isotopic variations in the mass of a biomolecule fragment or biomolecule. If correction for isotopic variations is desired (“YES” to test 1501), such correction 1510 is accomplished by any suitable method known in the art. In one embodiment, the intensities of the mass signals of the isotopic variants are conflated such that the intensity associated with the mass signal corresponding to the lowest mass isotopic variant is equal to the sum of the intensities of all the isotopic variants. In another embodiment, the areas of the mass signals of the isotopic variants are conflated such that the area associated with the mass signal corresponding to the lowest mass isotopic variant is equal to the sum of the areas of all the isotopic variants.

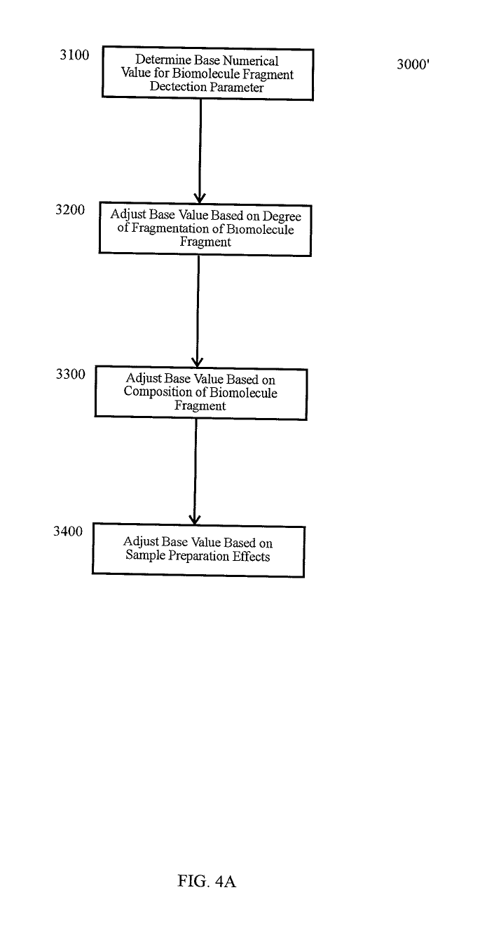

As discussed above, the matching of step 2000 compares a signal mass to the mass of a biomolecule fragment in a list which may be corrected for adducts or modifications. The signal mass is considered to “match” the biomolecule fragment mass if the masses are equal within a mass error range, referred to hereinafter as a “mass tolerance.” Referring to FIG. 3, various embodiments of matching mass signals to biomolecule fragments and identifying potential source biomolecules for the mass signals, which may be included in general step 2000, are shown. The investigator initially selects a biomolecule 2050 with known or predicted biomolecule fragments occurring in a list 2099 of biomolecule fragments known or predicted to be generated by the digestion and/or fragmentation of given biomolecules. The investigator selects a mass tolerance 2100. The mass tolerance may be a relative value or an absolute value. The precise value of the mass tolerance is determined based on, for example, the resolution of the mass spectrometry technique, accuracy of the mass spectrometry technique, the kinetics and/or dynamics of sample constituent fragmentation and ionization, and/or investigator convenience. In one embodiment, a relative mass tolerance in the range from about 2 ppm to about 300 ppm is selected. In another embodiment, a relative mass tolerance in the range of about 30 ppm to about 200 ppm is selected. In another embodiment, the mass tolerance is about 100 ppm.

As described above, the list may comprise one or more databases or elements of the list may be generated as desired, such as by on-the-fly or on-line calculation. In addition, the list need not contain all biomolecule fragments associated with a given biomolecule. Rather it is sufficient that the list contain only a subset of the biomolecules fragments associated with a biomolecule adequate for determining a likelihood for the presence or absence of the biomolecule under the sample preparation conditions and mass spectrometry technique employed. A mass signal is selected for comparison 2200 to a list 2099 of biomolecule fragments known or predicted to be generated by the digestion and/or fragmentation of given biomolecules.

Any mass signal may be selected for comparison 2200 to the list 2099 or selection may be limited to mass signals that meet certain criteria. In one embodiment, only mass signals with a signal intensity greater than a minimum intensity are selected (“YES” to test 2210). The signal mass of the selected mass signal is then compared 2300 to the mass of a biomolecule fragment in the list 2099 that correspond to those known or predicted to be generated for the biomolecule selected in step 2050. Alternatively, the investigator may omit selection of a biomolecule 2050 and the signal mass of the selected mass signal may be compared 2300 to the mass of any select biomolecule fragment in the list 2099.. If the signal mass is equal to the biomolecule fragment mass within the mass tolerance (“YES” to test 2310), the mass signal matches the biomolecule fragment, result 2301. Otherwise (“NO” to test 2310), the mass signal does not match the biomolecule fragment, result 2309. Comparison 2300 of the selected mass signal is then repeated for other biomolecule fragments 2350 of the select biomolecule 2050 and other mass signals 2355. Finally, additional biomolecules may be subjected to the same process of matching biomolecule fragments to mass signals (“YES” to test 2400”). The precise biomolecule fragments and biomolecules for which comparison 2300 is repeated, are determined based upon, for example, the nature of the sample, the biomolecule(s) suspected to be present in the sample, and/or investigator convenience. In one embodiment, the comparison 2300 is repeated for all biomolecule fragments and biomolecules in the list. In another embodiment, the comparison 2300 is repeated only for a subset of the biomolecule fragments and/or biomolecules in the list. For example, a subset may be chosen based on biomolecule molecular weight, and/or nature of the sample, such as the species from which the sample came.

Alternatively, an investigator may omit selection of a biomolecule 2050 for comparison. In one embodiment, a select mass signal 2200 is then compared to all select biomolecule fragments in list 2099 before the comparison is repeated for other mass signals 2355. In another embodiment, a biomolecule fragment in list 2099 is compared to all select mass signals before the comparison is repeated for other biomolecule fragments in the list. Accordingly, it is to be realized that the exact order of the comparisons and/or repetitive loops for biomolecule fragments, such as 2350, mass signals, such as 2355, and/or biomolecules with fragments in the list, such as 2400, is not central to the present invention. Rather, any suitable nesting of two or more of these repetitive loops is contemplated and suitable for the practice of the present inventions.

In one embodiment, for each repetition 2355 of comparison 2300, the mass tolerance may be changed (“YES” to test 2375), and as a result step 2100 is repeated, or the method may continue with the same mass tolerance (“NO” to test 2375). It is to be understood, however, that it is not central to the present invention whether repetition steps 2350 and 2355 are performed before or after repetition step 2400. For example, according to the methods of the present invention, a particular mass signals may be compared to a every biomolecule fragment in list 2099 of a every biomolecule before comparison 2300 is repeated for other mass signals.

Referring again to FIG. 3, once all repetitions 2400 are complete for all select biomolecules, potential source biomolecules are identified 2500 based on the mass signal-biomolecule fragment matches. As discussed above, a biomolecule can only be considered a potential source biomolecule if at least one mass signal matches a biomolecule fragment of the biomolecule (“YES” to test 2510). Otherwise (“NO” to test 2510), the biomolecule is not considered a potential source biomolecule, result 2509. In one embodiment, more than one mass signal must match the biomolecule fragments of a biomolecule before it is considered a potential source biomolecule (“YES” to test 2520). This embodiment reduces the number of biomolecules to be considered further,, i.e., implausible identifications of a biomolecule as a potential source biomolecule. For example, typically hundreds of proteins out of the approximately 4000 proteins of E. coli. match 3 peptides, i.e. biomolecule fragments, to a mass list containing about 100 masses. This embodiment is preferred when the biomolecule(s) suspected to be present in the sample and/or the biomolecule(s) of interest, are known or predicted to generate a large number of biomolecule fragments with a high likelihood, or probability, of detection. In this case, this embodiment reduces the incidence of biomolecules to be considered without significantly increasing false negatives. However, in other cases, for example, if a small number of biomolecule fragments are predicted, even biomolecules with a single matched biomolecule fragment may warrant further consideration.

In another embodiment, the mass signal-biomolecule fragment matches must satisfy other criteria in addition to the number of matches before the biomolecule associated with the matched biomolecule fragment is considered a potential source biomolecule. In one embodiment, each matched mass signal is required to satisfy a minimum signal intensity criterion (“YES” to test 2540). In another embodiment, each matched biomolecule fragment is required to satisfy a minimum biomolecule fragment detection parameter criterion (“YES” to test 2550). The determination of a biomolecule fragment detection parameter associated with a biomolecule fragment of a given biomolecule is discussed below. In another embodiment, the mass signal-biomolecule fragment match(es) is required to satisfy both mass signal intensity and biomolecule fragment detection parameter criteria. Accordingly, a biomolecule is considered a potential source biomolecule of the mass signals matched to its associated biomolecule fragments, result 2501, if a minimum number of mass signals match the biomolecule fragments of the biomolecule (“YES” to test 2510 and “NO” to either test 2520 or 2530) and the mass signal-biomolecule fragment matches satisfy any additional selected criteria, such as tests 2540 to 2555.

According to the methods of the present invention, the identification 2500 is repeated 2600 for other select biomolecules of the list 2099. The precise biomolecules selected are determined based on, for example, the sample under investigation, the potential source biomolecule(s) earlier identified, and/or investigator convenience. For example, only biomolecules where at least one associated biomolecule fragment matches a mass signal, may be selected, and as a result, obviate the need to perform step 2510.

In one embodiment, the general step 2000 is repeated employing in comparison 2300 a list 2099 composed of the biomolecule fragments known or predicted to be generated by the digestion and/or fragmentation of the potential source biomolecules identified in a prior iteration of step 2000. Typically in this embodiment, both the mass tolerance selected in step 2100, and the criteria of steps 2520 to 2555, are relaxed to match previously unmatched mass signals to a biomolecule and thereby identify potential source biomolecule fragments for a greater number of mass signals.

As discussed above, step 3000 determines a numerical value, referred to as a biomolecule fragment score, which quantifies the significance of the match of a mass signal to a biomolecule fragment of a potential source biomolecule. The biomolecule fragment score is proportional to the associated biomolecule fragment detection parameter. In one embodiment, the biomolecule fragment score is also a function of the closeness of the match as expressed by the difference between the signal mass and matched biomolecule fragment's theoretical mass. In one embodiment, the biomolecule fragment score is directly proportional to the biomolecule fragment detection parameter. Accordingly, in this embodiment where a high biomolecule fragment detection parameter indicates a high likelihood of detection, the higher the biomolecule fragment score, the greater the significance of the match of a mass signal to that biomolecule fragment for a given biomolecule. However, it is not central to the present invention whether an increasing, decreasing, positive or negative biomolecule fragment score corresponds to a greater significance for the match. Rather, the methods of the present invention require only that the significance associated with a biomolecule fragment score is substantially a monotonic function of the biomolecule fragment score.

In one embodiment, the biomolecule fragment score is directly proportional to the biomolecule fragment detection parameter and the corresponding matched mass signal intensity and inversely proportional to the relative difference between the signal mass and matched biomolecule fragment mass, as illustrated in equation 1:

$${SN}_{pp} ∝ \frac{( {I}_{ms} ) \times ( {C}_{dp} )}{( ( \vert {m}_{ms} - {m}_{pp} \vert / {m}_{ms} ) + {ppm}_{min} )} \qquad ( 1 )$$

where Sₚₚ is the biomolecule fragment score, Iₘₛ is the intensity of the mass signal in the mass spectrum, C_{dp} is the biomolecule fragment detection parameter, mₘₛ is the corresponding signal mass, mₚₚ is the mass of the matched biomolecule fragment, and ppmₘᵢₙ is the minimum ppm error to be considered. The relative mass difference is preferably expressed in ppm. In this embodiment, the term ppmₘᵢₙ provides a lower limit for the denominator of equation 1 to prevent division of the numerator by zero. Preferably, the value of ppmₘᵢₙ is set to the mass accuracy of the mass spectrometer, because a mass difference less than the limit of accuracy of the mass spectrometer is statistically unreliable. However, it should be understood by one of ordinary skill in the art that whenever the value of a function, such as the biomolecule fragment score or the biomolecule score, is inversely proportional to one of its variables, such as the difference between the signal mass and matched biomolecule fragment mass, a lower limit should be set for this variable by some method to prevent division by zero. In another embodiment, the biomolecule fragment score is directly proportional to the biomolecule fragment detection parameter and the corresponding matched mass signal intensity and inversely proportional to the absolute difference between the signal mass and matched biomolecule fragment mass.

In another embodiment, the biomolecule fragment score is directly proportional to the corresponding biomolecule fragment detection parameter and inversely proportional to the absolute difference between the signal mass and the matched biomolecule fragment mass. In still another embodiment, the biomolecule fragment score is directly proportional to the corresponding matched mass signal intensity and inversely proportional to the difference between the signal mass and the matched biomolecule fragment mass.

In another embodiment, the biomolecule fragment score is dimensionless and directly proportional to the biomolecule fragment detection parameter and the corresponding matched mass signal intensity and inversely proportional to the relative difference between the signal mass and matched biomolecule fragment mass, as illustrated in equation 2:

$${SN}_{pp} ∝ \frac{( {I}_{ms} {/ \sum }_{i} {I}_{ms} ) \times ( {C}_{dp} / {\sum }_{i} {C}_{dp} )}{( ( \vert {m}_{ms} - {m}_{pp} \vert / {m}_{ms} ) + {ppm}_{min} )} \qquad ( 2 )$$

where SNₚₚ is the dimensionless biomolecule fragment score, Iₘₛ is the intensity of the mass signal in the mass spectrum, ΣᵢIₘₛ is the total matched mass signal intensity, C_{dp} is the biomolecule fragment detection parameter, ΣᵢC_{dp} is the sum of all the biomolecule fragment detection parameters, mₘₛ is the corresponding signal mass, mₚₚ is the mass of the matched biomolecule fragment, and ppmₘᵢₙ is the minimum ppm error to be considered. The relative mass difference is preferably expressed in ppm.

The quantification of the significance the match 3000 takes into account the variation in the probability of detecting various biomolecule fragments. Biomolecule fragment detection likelihood can vary, for example, with mass spectrometry technique, degree of biomolecule fragmentation, biomolecule digest, amino acid(s) of a biomolecule fragment, sequence of the biomolecule fragment, and position of the biomolecule fragment in a biomolecule sequence. Where a biomolecule fragment detection likelihood is considered, a numerical value is determined and assigned to each matched biomolecule fragment of selected potential source biomolecules that is a measure of the likelihood of detecting that biomolecule fragment as a fragment and/or digestion product of the biomolecule within a given digest and/or fragmentation and using a given mass spectrometry technique. This numerical value is referred to as the “biomolecule fragment detection parameter.”

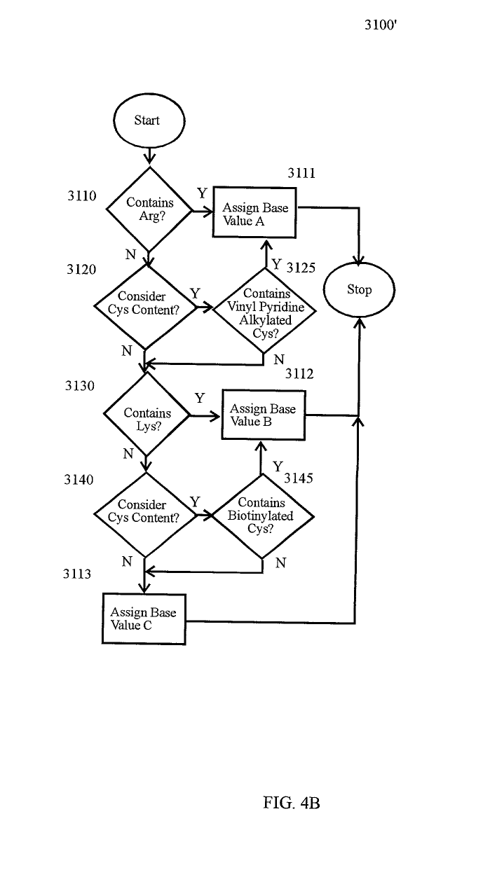

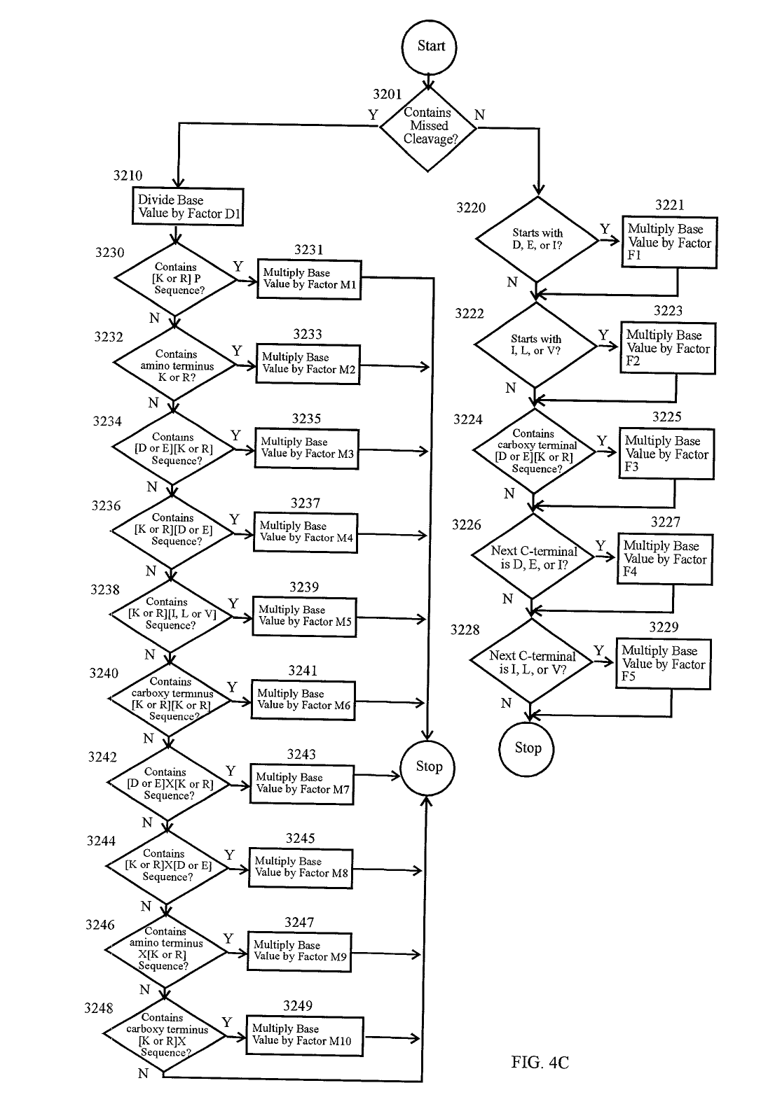

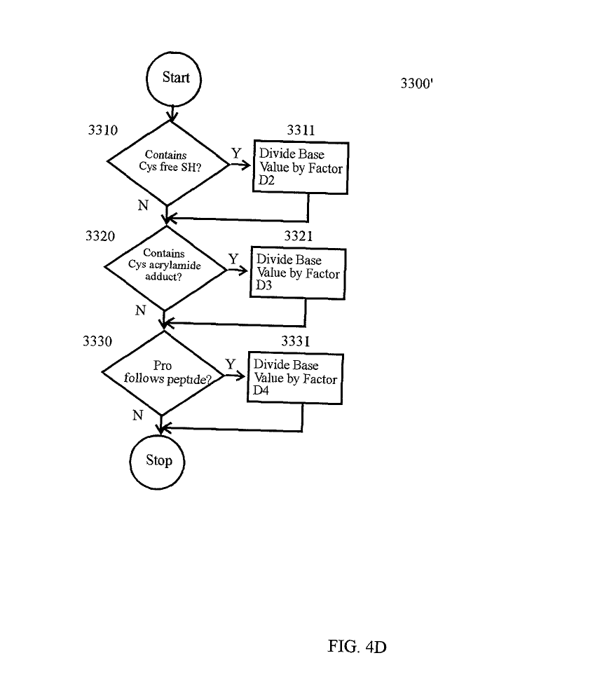

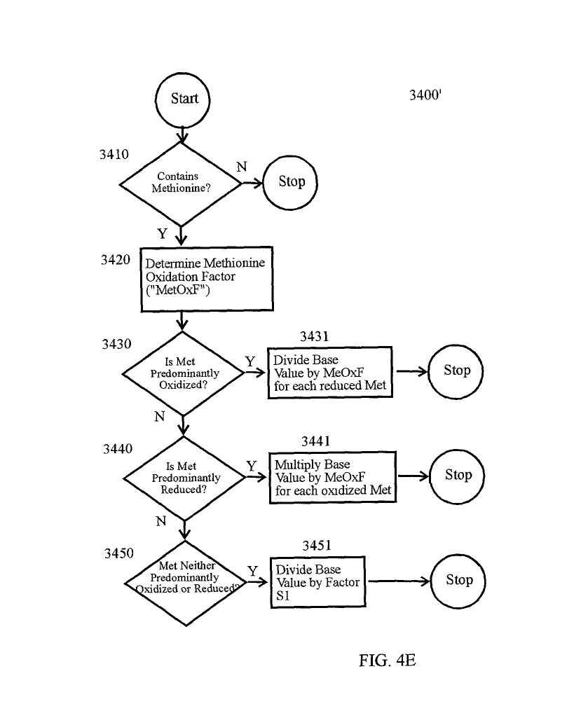

Referring to FIG. 4A, in one embodiment the biomolecule fragment detection parameter determination 3000′ comprises determining a base value 3100 which is subsequently adjusted for the extent, or degree, of fragmentation of the biomolecule fragment 3200, influences on the fragmentation and/or detection likelihood based on the composition and/or sequence of the biomolecule fragment 3300, and/or influences on the detection likelihood that arise from sample preparation 3400. While FIG. 4A illustrates the modification of the base numerical value to obtain a biomolecule fragment detection parameter in a specific order, it is to be understood that the order in which steps 3200 to 3400 are performed is not central to the present invention and may vary. In addition, certain of these steps may be eliminated.

In one embodiment, the effects of the sample digest and/or fragmentation, mass spectrometry technique, and composition of the biomolecule fragment are considered in determining a biomolecule fragment detection parameter. An underlying principal to determining a biomolecule fragment detection parameter is that the numerical values of the parameters reflect the general relative mass signal intensity relationships between biomolecule fragments, and/or the fraction of a biomolecule fragment generally observed, in a mass spectrum of the sample or related samples that arise from differences in biomolecule fragment sequence and chemistry of the biomolecule fragmentation and/or digestion. The relative intensity relationship and fraction of a biomolecule fragment generally observed can be determined for any given protein digestion and any given mass spectrometry technique. For example, a biomolecule fragment detection parameter for a peptide can be determined for any peptide given its sequence and neighboring amino acids in the protein.

The general relative intensity relationship can be determined by comparison of measured biomolecule fragment mass signal intensities generated from a sample of known biomolecule(s). Alternatively, the general relative intensity relationship can be determined from comparison of biomolecule fragment mass signal intensities predicted for a sample. Likewise, the fraction of a biomolecule fragment generally observed can be determined by comparison of measured biomolecule fragment mass signals generated from a sample of known biomolecule(s). Alternatively, the fraction of a biomolecule fragment generally observed can be predicted for the biomolecule fragment of a given biomolecule. The relative intensity relationship and fraction of a biomolecule fragment generally observed may be determined, for example, from published data or from data obtained by the investigator. An example of the latter such determination is illustrated below in Example 1 for an analysis employing a trypsin digest of proteins and a MALDI-TOF mass spectrometry technique.

In another embodiment, numerical values for the biomolecule fragment detection parameters are determined using a sample of known composition and parameters representing properties of the biomolecule fragmentation and/or detection that influence the detection likelihood of potential biomolecule fragments. Such properties, include but are not limited to, degree of fragmentation of the biomolecule fragment, composition of the biomolecule fragment, and sample preparation effects. In this embodiment, initial numerical values for these parameters are varied until the biomolecule(s) known to be present in the sample obtain a biomolecule score(s) indicative of a likelihood of being present that is significantly greater than that for the biomolecule score(s) of background matches. By background match is meant a match of one or more mass signals to a biomolecule in a list which is known not to be present in a sample. The above variation and value determination may, for example, be accomplished by solution of simultaneous equations, with the aid of a neural network, and/or genetic algorithm. In addition, the value of one or more parameters may be fixed, determined experimentally and/or derived from experimental data.

For example, in one embodiment where the biomolecules of interest are proteins, the methods of the present invention take into account that trypsin typically does not cleave every peptide bond equally well. As a result, some of the resulting biomolecule fragments, i.e. peptides, will contain one or more missed cleavages. The present invention may take into consideration both single and multiple missed cleavages. For example, trypsin rarely cleaves either lysine-proline (Lys-Pro or K-P) or arginine-proline (Arg-Pro or R-P) bonds. Trypsin cleavage of Lys and Arg bonds that follow or precede glutamic acid (Glu or E) or Lys, N-terminal Lys and Arg bonds, as well as C-terminal double basics, is also generally poor. Further, this embodiment typically takes into account that for mass spectrometry techniques that employ MALDI, tryptic peptides that contain (Arg or R) are detected more easily than peptides that contain (Lys or K) but not Arg. In another embodiment, the effect of sample preparation is considered in determining the biomolecule fragment detection parameter of proteins peptides, for example, depending on sample preparation, methionines are often found reduced, or oxidized to the sulfoxide, or sulfone. In another embodiment, the methods of the invention take into account that cysteine (Cys) containing peptides are usually poorly detected if they have not been alkylated. Further, it is to be realized that other properties are potential influences on the fragmentation and/or detection of biomolecules and their fragments. For example, where the biomolecules of interest comprise proteins, hydrophobicity, ionization yield and molecular weight of the biomolecule fragments, may potentially influence fragmentation and/or detection.

Referring to FIGS. 4B-E, one embodiment for determining biomolecule fragment detection parameters is shown for an analysis of the proteins of a sample that employs a trypsin digest and a MALDI mass spectrometry technique to obtain the sample mass spectrum. The biomolecule fragment detection parameter is equal to a base numerical value (determined in step 3100) that is primarily dependent on the presence of Arg or Lys and mass spectrometry technique, modified by factors (determined in steps 3200 to 3400) that are primarily dependent on the chemistry of the digest, degree of digestion of the peptide sample preparation and position of the peptide in the sequence of the associated protein (i.e., biomolecule). Accordingly, the methods of the present invention take into consideration not only single missed cleavages in determining biomolecule fragment detection parameters, but may also take into consideration multiple missed cleavages and the composition of the biomolecule fragment with missed cleavages.

In one embodiment, the base numerical value is determined primarily by whether the peptide contains certain amino acids . A base value of eight is assigned 3180 to peptides with an Arg (“NO” to test 3110, indicating result 3111). A peptide with a Lys, but no Arg, (“YES” to test 3110) is assigned a base value of three, action 3130, if the peptide does not contain a vinyl pyridine alkylated Cys (“NO” to test 3115). If a Lys peptide, which does not contain an Arg, also contains a vinyl pyridine alkylated Cys (“YES” to test 3115), a base value of eight is assigned 3180 to the biomolecule fragment detection parameter of the peptide.

Referring to FIG. 4C, various embodiments 3200′ that take into account the degree of biomolecule fragment fragmentation are shown. The primary consideration is whether the peptide contains a missed cleavage (test 3201). Step 3200′ takes into account that the amino acids of a peptide can effect the activity of a trypsin digest. Certain amino acids and amino acid sequences are known to reduce the activity of trypsin at potential trypsin cleavage sites. In the case where the peptide is a fully digested, i.e., no missed cleavages, (“NO” to test 3201), the base value of the biomolecule fragment detection parameter of the peptide is modified if: (1) the peptide starts with an Asp, Lys, leucine (Leu or L), isoleucine (Ile or I) or valine (Val or V), (“YES” to test 3220 or 3222); (2) the next amino acid C-terminal to the peptide is Glu (E), aspartic acid (Asp or D), Ile, Leu, or Val (“YES” to tests 3226 or 3228); or (3) contains a carboxy terminal Asp-Lys, Glu-Lys, Asp-Arg, or Glu-Arg sequence (“YES” to test 3224). Since the effect of these sequences on trypsin activity is generally not large, the base value is reduced (results 3221, 3223, 3225, 3227 and/or 3229) only by about 2.5% to 15% for each such sequence. Accordingly, the values of factors F1 to F5, in one embodiment, are respectively about 0.9, 0.95, 0.9, 0.9, and 0.95.

Step 3200′ may further take into account that trypsin does not always cleave all Arg-Lys bonds in a protein (“YES” to test 3201). As a result, some biomolecule fragments, i.e. peptides, of a protein will contain missed cleavages. Peptides containing missed cleavages that do not fall into one of the categories described below generally account for a minor portion of a corresponding protein's mass spectral signal intensity. Accordingly, the base numerical value for all peptides with missed cleavages is initially reduced by a factor D1 (action 3210), with missed cleavages in the categories described below accounted for later. In one embodiment, D1 is a value in the range from about 10 to 100. In another embodiment, D1 is a value in the range from about 25 to 75. In another embodiment, D1 is about 50.

In other embodiments, step 3200′ further takes into account that the likelihood a cleavage is missed also depends on the sequence of amino acids adjacent to or within the missed cleavage peptide (test and results 3230 to 3249). For example, if the missed cleavage peptide contains a Arg-Pro or Lys-Pro missed cleavage (“YES” to test 3230) the missed cleavage peptide is generally treated as a fully digested peptide. As a result, the base value is multiplied by a factor M1 (result 3231) which is typically substantially equal to D1.

Missed cleavage peptides which contain a Lys or Arg at the amino terminus (“YES” to test 3232), a Asp-Lys, Asp-Arg, Glu-Lys, or Glu-Arg sequence (“YES” to test 3234), or a Lys-Asp, Lys-Glu, Arg-Asp, Arg-Glu sequence (“YES” to test 3236) are treated almost as if they were fully digested peptides. Accordingly, the base value is multiplied by a factor M2, M3, or M4, as appropriate. In one embodiment, the values of each of M2-M4 fall in the range from about one-fourth to one-half D1. In one embodiment, M2 to M4 are each equal to about two-fifths D1.

Missed cleavage peptides which contain a Lys-Ile, Lys-Leu, Lys-Val, Arg-Ile, Arg-Leu, or Arg-Val sequence (“YES” to test 3238) have their base values multiplied by M5 (result 3239). Factor M5 generally has a value in the range from about one-tenth to one-fourth D1. In one embodiment, M5 is about one-fifth D1. Missed cleavage peptides which contain a Lys-Arg, Lys-Lys, Arg-Arg or Arg-Lys sequence at the carboxy terminus (“YES” to test 3240) have their base values multiplied by M6 (result 3241). Missed cleavage peptides which contain a Asp-X-Lys, Asp-X-Arg, Glu-X-Lys, or Glu-X-Arg sequence, where X is any one amino acid, (“YES” to test 3242), or a Lys-X-Asp, Lys-X-Glue, Arg-X-Asp, Arg-X-Glu sequence (“YES” to test 3244) have their base values multiplied by, respectively, M7 (result 3243) or M8 (result 3245). Such missed cleavage peptides typically show a somewhat greater likelihood of detection than missed cleavage peptides in general. Accordingly, in one embodiment, the values of each of M5-M7 fall in the range from about one-twentieth to one-fourth D1. In one embodiment, M5 to M7 are each equal to about one-tenth D1.

Missed cleavage peptides which contain a X-Lys or X-Arg sequence at the amino terminus, where X is any one amino acid, (“YES” to test 3246), or those which contain a Lys-X-Lys, Lys-X-Arg, Arg-X-Lys or Arg-X-Arg sequence at the carboxy terminus (“YES” to test 3248) have their base values multiplied respectively, M9 (result 3247) or M10 (result 3249). Such missed cleavage peptides typically show a slightly greater likelihood of detection than missed cleavage peptides in general. Accordingly, in one embodiment, the values of each of M9 and M10 fall in the range from about one-fiftieth to one-tenth D1. In one embodiment, M9 and M10 are each equal to about three-fiftieths D1.

Referring to FIG. 4D, various embodiments for taking into consideration the effect of biomolecule fragment composition on the likelihood of biomolecule fragment detection are shown. Step 3300′ takes into account that trypsin cleaves Arg-Pro and Lys-Pro bonds so poorly that if Pro is C-terminal to the peptide, the peptide is usually less abundant than the longer peptide containing the intact Lys-Pro or Arg-Pro bond, which is commonly classified as being a terminal digestion product by most protein chemsits. Thus, if the peptide has a following Pro (“YES” to test 3330), the base value is divided by a factor D3 (result 3331). In one embodiment, where the biomolecule fragment detection likelihood increases with the biomolecule fragment detection parameter, D3 is greater than one and has a value in the range in the range from about 10 to 100. In another embodiment, D3 has a value of about 50. This decrease (result 3331) reflects the relatively poor cleavage by trypsin of Arg-Pro and Lys-Pro bonds.

Step 3300′ may further take into account that the presence of Cys in a peptide, especially unalkylated Cys, can reduce the recovery of the peptide. Accordingly, if a peptide contains a Cys free SH (“YES” to test 3310), the base value of the biomolecule fragment detection parameter is divided by a factor D2, action 3311. Similarly, if a peptide contains a Cys acrylamide adduct (“YES” to test 3320), the base value of the biomolecule fragment detection parameter is divided by a factor D3, action 3321, to reflect the rather low efficiency of alkylation of Cys by acrylamide in most gel systems Accordingly, D2 and D3 typically have values in the range from about 2 to 50. In one embodiment, D2 and D3 each have a value of about 10.

Referring to FIG. 4E, an embodiment for taking into consideration an effect of sample preparation on biomolecule fragment detection likelihood is shown. Step 3400′ takes into account that the predominant oxidation state of methionine (Met) depends on the treatment of the sample and affects the detection likelihood of a peptide. It is typically found that in some digests of known proteins, the majority of methioines are oxidized, whereas in other cases, the majority of methionines are reduced. To take this into consideration, a Methionine Oxidation Factor (MetOxF) is determined. In one embodiment, the MetOxF is assigned a value of 4 if the reduced form of most peptides is usually ¼ the intensity of the oxidized form of the same peptide, and (by convention) a value of 0.25 if the oxidized form of most peptides is usually ¼ the intensity of the reduced form of the same peptide (action 3420). Thus, if the peptide contains Met (“YES” to test 3410) and methionine is predominantly oxidized, (“YES” to test 3430), the base value of the biomolecule fragment detection parameter is divided by the MetOxF for each reduced methionine in the peptide (action 3431). If no reduced methionine is present in the peptide, the base value is not modified. Similarly, in the case where the peptide contains Met (“YES” to test 3410) and methionine is predominantly reduced, (“NO” to test 3430, “YES” to test 3440), the base value of the biomolecule fragment detection parameter is multiplied by the MetOxF for each oxidized methionine in the peptide (action 3441). If no oxidized methionine is present in the peptide the base value is not modified. Further, if the methionines are neither predominantly oxidized or reduced, MetOxF equal to one, the base value is divided by a factor S1. In one embodiment, S1 is a value in the range of about 1 to 5. In another embodiment, the value of S1 is about 2.

Determinations of biomolecule fragment detection parameters, employing the principals described herein, can be made for other digests and/or mass spectrometry techniques. For example, determinations made for an analysis of proteins employing a trypsin digest and an electrospray ionization technique coupled with a TOF mass spectrometer, have shown that the contribution to the total matched mass signal intensity for Arg containing peptides and Lys containing peptides is approximately equal. Accordingly, the base value of the biomolecule fragment detection parameters for Arg and Lys containing peptides under such analysis conditions should be substantially equal. Further, it is to be realized that although the above examples describe determination of biomolecule fragment detection parameters for a specific protein digest and mass spectrometry technique, detection parameters can be further “customized” to a specific sample or class of samples, or even, to a specific sample preparation technique. Accordingly, it is to be realized that biomolecule fragment detection parameters can reflect the relative intensity relationships of peptide mass signals and fraction of a peptide observed under a very specific set of sample analysis conditions, properties, and techniques, or under the broader categories of sample digest and mass spectrometry technique. While the biomolecule fragment detection parameters of various peptides have been given specific values, in no case are any of these values central to the functioning of the present invention. Rather, the specific values can be selected to reflect, for example, the detection efficiency of the mass spectrometry technique, the chemistry of the study, and/or a model of the detection probability of a biomolecule fragment signal.

Returning to general step 3000, in another embodiment, the biomolecule fragment score is directly proportional to the corresponding mass signal intensity and inversely proportional to the relative difference between the signal mass and the matched biomolecule fragment mass, as illustrated in equation 3:

$${S}_{pp} ∝ \frac{{I}_{ms}}{( ( \vert {m}_{ms} - {m}_{pp} \vert / {m}_{ms} ) + {ppm}_{min} )} \qquad ( 3 )$$

where Sₚₚ is the biomolecule fragment score, Iₘₛ is the intensity of the mass signal in the mass spectrum, mₘₛ is the corresponding signal mass, and mₚₚ is the mass of the matched biomolecule fragment, and ppmₘᵢₙ is the minimum ppm error to be considered.

As discussed above, step 4000 determines a numerical value referred to as a biomolecule score which quantifies the likelihood that a potential source biomolecule identified in step 2000 is present in the sample. The biomolecule score is a function of the biomolecule fragment score(s) of the matched biomolecule fragment(s) of the biomolecule. Accordingly, the biomolecule score, and as a result, the likelihood that the potential source biomolecule is present in the sample, depends on the likelihood of detecting the associated matched biomolecule fragment(s).

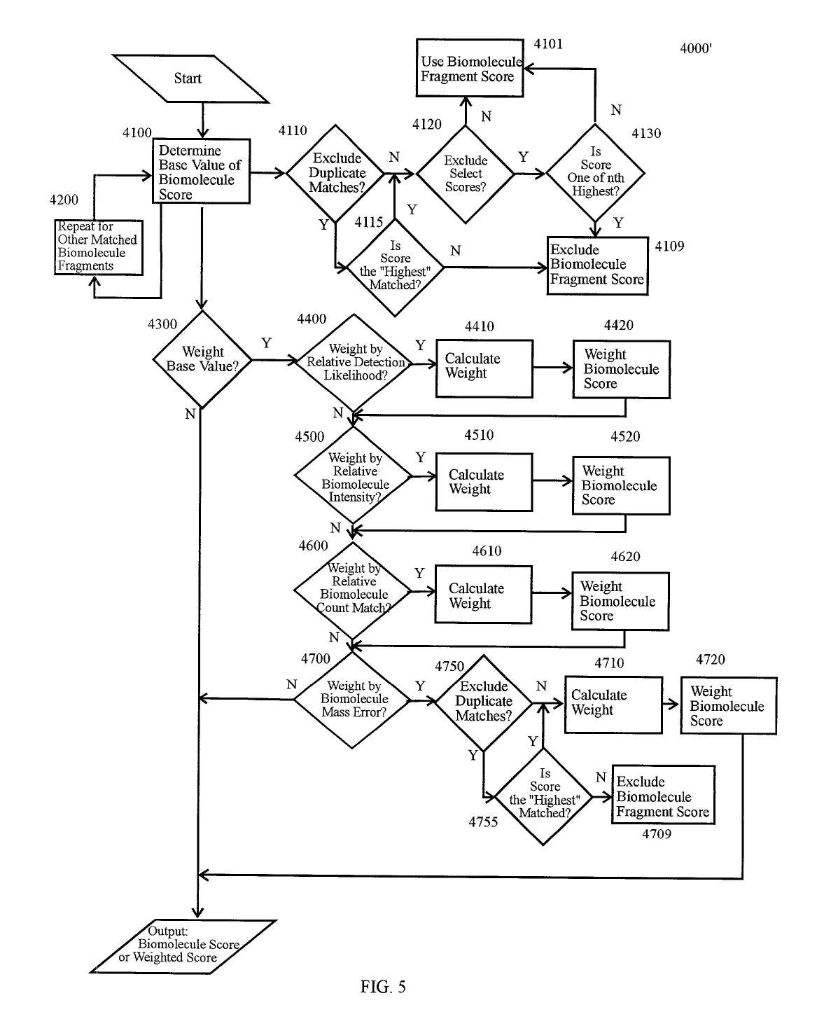

Referring to FIG. 5, various embodiments for determining a biomolecule score are shown. In one embodiment, the biomolecule score is equal to a base numerical value (determined in step 4100) weighted by another numerical value or values (see step 4300 and steps 4400 to 4700). In another embodiment, the biomolecule score is equal to the base numerical value determined in step 4100. Accordingly, it is to be understood that weighting is not central to the present invention. In addition, while FIG. 5 illustrates weighting the base value in a specific order, it is to be understood that the order in which steps 4400 to 4700 are performed is not central to the present invention and may vary, and that one or more steps 4400 to 4700 may be eliminated. Further, in the following discussion of FIG. 5 reference is made to the “highest” biomolecule fragment score. In the interest of a concise discussion of FIG. 5, the “highest” biomolecule fragment score is meant to refer to the biomolecule fragment score that corresponds to the match with the greatest significance. However, as discussed above, it is to be understood that it is not central to the present invention whether an increasing, decreasing, positive or negative biomolecule fragment score corresponds to a greater significance for the match.

The biomolecule score base numerical value is determined primarily by the biomolecule fragment scores of the matched biomolecule fragments of the biomolecule. In one embodiment, the methods of the present invention limit each mass signal to matching only one biomolecule fragment of each biomolecule. In this embodiment, duplicate matches to a biomolecule are excluded from the biomolecule score determination (“YES” to test 4110) of that biomolecule. Exclusion of duplicate matches prevents such matches from possibly inflating biomolecule scores. In one embodiment, only the “highest” biomolecule fragment scores associated with each match to a given mass signal are used to determine the biomolecule score of the corresponding biomolecule (“YES” to test 4115). In another embodiment, contributions from duplicate matches are not excluded (“NO” to test 4110). However, it is typically preferable to exclude duplicate matches when, for example, the mass signal is one of the most intense signals in te mass spectrum and a small number of matches were required at step 2520.

In either embodiment (i.e., “YES” or “NO” to test 4110), additional biomolecule fragment scores may be excluded from the biomolecule score determination. Preferably, select biomolecule fragment scores of the matched biomolecule fragments are excluded from the determination of the biomolecule score (“YES” to test 4120). In one embodiment, one or more of the “highest” biomolecule fragment scores (“YES” to test 4130) are excluded (or constrained to make a limited contribution to the biomolecule score) to ensure that a statistically unreliable number of biomolecule fragment matches associated with high intensity mass signals and/or an associated biomolecule fragment(s) a with significant biomolecule fragment detection parameter(s) do not dominate the biomolecule score. In another embodiment, the base numerical value is a function of the biomolecule fragment scores of all the matched biomolecule fragments of the associated biomolecule (“NO” to test 4120). Inclusion of all the biomolecule fragment scores is preferred when the biomolecule(s) suspected to be present in the sample and/or the biomolecule(s) of interest, are known or predicted to generate few biomolecule fragments upon digestion and/or fragmentation that have a reasonable probability or likelihood of detection, for example, from proteins isolated from the bottom of an SDS gel. In this case, inclusion of all the biomolecule fragment scores prevents underestimating the probability that such biomolecules are present in the sample. The determination 4100 is repeated 4200 for other matched biomolecule fragments of the biomolecule to determine a base value of the biomolecule score.

In one embodiment, (“YES” to test 4300) the biomolecule score base value is weighted by at least one numerical value that reflects supplementary information on the likelihood that the potential source biomolecule is present in, or absent from, the sample. In one embodiment, the base value is weighted by a value, referred to as a “relative biomolecule detection parameter”, that reflects the relative detection probability or likelihood of a potential source biomolecule (“YES” to test 4400). In one embodiment, the relative biomolecule detection parameter is calculated 4410 by (1) summing the biomolecule fragment detection parameters of each of the biomolecule fragments that match to determine a potential source biomolecule score, (2) summing two or more of the biomolecule fragment detection parameters of the biomolecule fragments that can be generated from the biomolecule in question to determine a “signal biomolecule fragment score”; and (3) dividing the potential source biomolecule score by the signal biomolecule fragment score. In one embodiment, the signal biomolecule fragment score comprises the sum of all of the biomolecule fragment detection parameters of the biomolecule fragments that can be generated from the biomolecule in question under the digestion and/or fragmentation conditions used. Weighting by the relative biomolecule detection parameter is preferred when the biomolecule(s) suspected to be present in the sample and/or the biomolecule(s) of interest; are known or predicted to generate relatively few biomolecule fragments upon digestion and/or fragmentation that have a reasonable probability or likelihood of detection. Accordingly, weighting by the relative biomolecule detection parameter is generally preferred when a biomolecule suspected to be present in the sample and/or the biomolecule of interest is a small biomolecule.

In another embodiment, the base value is weighted by a value, referred to as a “relative biomolecule intensity”, that reflects the relative intensity of a potential source biomolecule (“YES” to test 4500). In one embodiment, the relative biomolecule intensity is calculated 4510 by: (1) summing the intensities of the mass signals associated with the matched biomolecule fragments to determine a “potential source biomolecule intensity score”; (2) summing two or more of the signal intensities of the mass signals to determine a “signal intensity score;” and (3) dividing the potential biomolecule intensity score by the signal intensity score. In one embodiment, the signal intensity score comprises the sum of all of the signal intensities of all the mass signals selected in step 2200. In another embodiment, the signal intensity score comprises the sum of all of the signal intensities of all the mass signals of the mass spectrum or the corrected mass spectrum. Weighting by the relative biomolecule intensity is preferred when the biomolecule(s) suspected to be present in the sample and/or the biomolecule(s) of interest are known or predicted to generate many biomolecule fragments upon digestion and/or fragmentation that have a reasonable probability or likelihood of detection. Accordingly, weighting by the relative biomolecule intensity is generally preferred when a biomolecule suspected to be present in the sample and/or the biomolecule of interest is a large biomolecule, a biomolecule present in high concentration and/or possess many sites at which the biomolecule is readily fragmented. For example, weighting by the relative biomolecule intensity is generally preferred when the biomolecules of interest are proteins and where the protein(s) of interest possess many amino acid bonds which are readily cleaved by the digestion agent employed.

In another embodiment, the base value is weighted by a value, referred to as a “relative biomolecule match count,” that reflects the relative significance of the number of mass signals matched to a potential source biomolecule (“YES” to test 4600). In one embodiment, the relative biomolecule match count is calculated 4610 by: (1) summing the number of matched biomolecule fragments to determine a “biomolecule fragment count” of the potential source biomolecule; and (2) dividing the biomolecule fragment count by the number of biomolecule fragments that can be generated from the biomolecule in question under the digestion and/or fragmentation conditions used. Weighting by the relative biomolecule match count is preferred when the biomolecule(s) suspected to be present in the sample and/or the biomolecule(s) of interest are known or predicted to generate few biomolecule fragments upon digestion and/or fragmentation that have a reasonable probability of detection. Accordingly, weighting by the relative biomolecule match count is generally preferred when a biomolecule suspected to be present in the sample and/or the biomolecule of interest is a biomolecule with few fragments and/or a biomolecule present in low concentration.

In another embodiment, the base value is weighted by a value, referred to as a “biomolecule mass error,” that reflects the overall closeness of the mass signal-biomolecule fragment matches for a potential source biomolecule (“YES” to test 4700). The biomolecule mass error is a function of the difference between the signal mass and matched biomolecule fragment mass. The mass difference can be either a relative or an absolute difference. In addition, this mass difference may itself be weighted by the mass signal intensity of the matched signal masses. Preferably, the mass difference is the relative mass difference expressed in ppm. In one embodiment, the biomolecule mass error is calculated 4710 by summing the relative difference between the signal mass and matched biomolecule fragment mass, weighted by the intensity of the mass signal, for each matched biomolecule fragment of the biomolecule, as illustrated in equation 4:

$$IBME = ( {\sum }_{{m}_{ms} , {m}_{pp}} {I}_{ms} \times {10}^{6} \times ( ( {m}_{ms} - {m}_{pp} ) / {m}_{ms} ) ) / ( {\sum }_{i} {I}_{ms} ) \qquad ( 4 )$$

where IBME is the intensity weighted biomolecule mass error, Iₘₛ is the intensity of the mass signal in the mass spectrum, ΣᵢIₘₛ is the total matched mass signal intensity, mₘₛ is the corresponding signal mass, mₚₚ is the mass of the matched biomolecule fragment, and the factor of ten to the sixth is used to here to explicitly express the mass difference in ppm. Mass error weighting is preferred when a large number of signals close to the detection limit match the biomolecule fragment masses. In such a case, the most significant signals are typically the most intense signals; correspondingly, some of the weaker signals are less accurate because mass accuracy generally decreases as signal intensity decreases. Further, duplicate matches may also be excluded from the biomolecule mass error determination (“YES” to test 4750). Exclusion of duplicate matches prevents such matches from possibly inflating the biomolecule mass error, and as a result, underestimating the closeness of the mass signal-biomolecule fragment matches for a potential source biomolecule. In another embodiment, only the “highest” biomolecule fragment score associated with the match to a given mass signal is used to determine the mass error of the associated biomolecule (“YES” to test 4755).

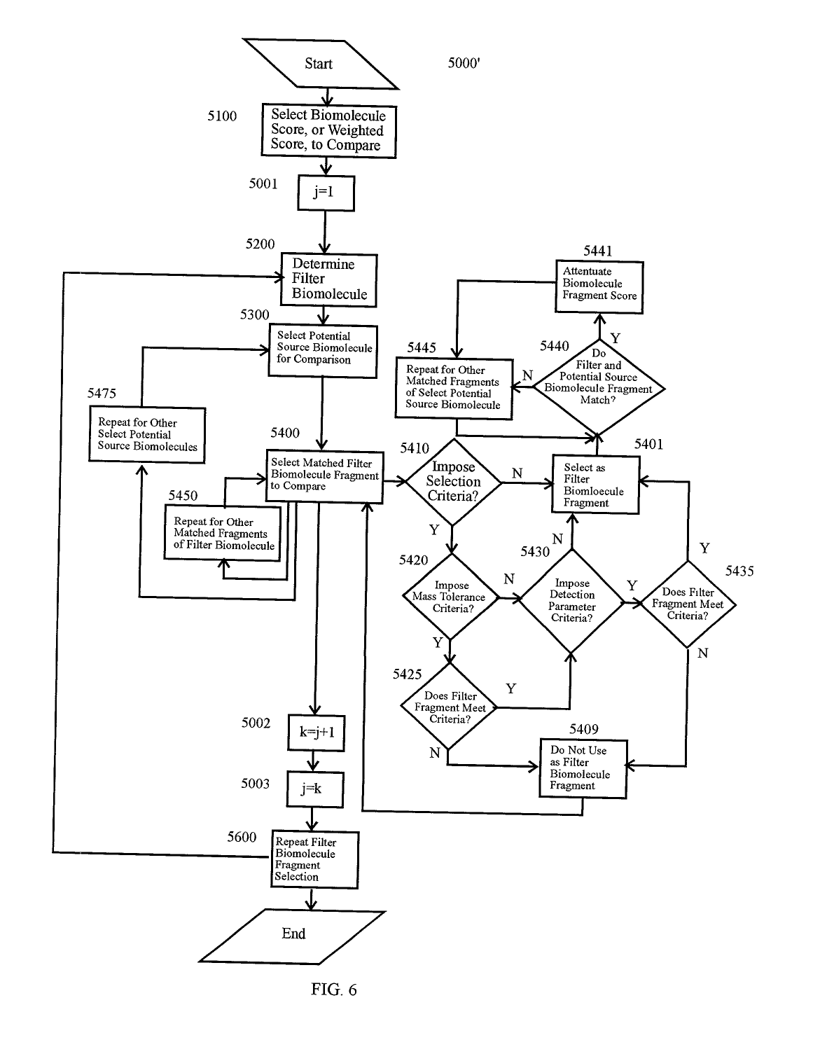

As discussed above, step 5000 determines the likelihood of the presence or absence of a biomolecule in the sample based on the biomolecule scores, or weighted biomolecule scores, of the potential source biomolecules. The biomolecule scores, or weighted biomolecule scores, determined according to the methods illustrated in FIG. 5, are compared to determine whether it is likely a biomolecule is present in, or absent from, the sample. One embodiment for determining the likelihood of the presence or absence of a biomolecule in the sample is illustrated in FIG. 6. In the following discussion of FIG. 6 reference is made to the “highest” biomolecule score, or weighted biomolecule score. In the interest of a concise discussion of FIG. 6, the phrase “highest” biomolecule score, or weighted biomolecule score, is meant to refer to the score that corresponds to the highest likelihood that the potential source biomolecule is present in the sample. However, as discussed above, it is not central to the present invention whether an increasing, decreasing, positive or negative biomolecule score corresponds to an increasing probability or likelihood that the potential source biomolecule is present.

Referring to FIG. 6, a biomolecule score, or weighted biomolecule score, on which to base the determination of the likelihood of the presence or absence of a biomolecule in the sample is selected in step 5100. In the interest of a concise discussion of FIG. 6, reference will generally be made only to biomolecule scores, however, it is to be understood that such reference includes weighted biomolecules scores. An iteration parameter, j, which serves simply to facilitate the description of the methods, is assigned an initial value of one, action 5001. The potential source biomolecule with the jth “highest” biomolecule score is selected as the “filter biomolecule” for that iteration 5200. For example, in the first iteration, j=1, the potential source biomolecule with the “highest” biomolecule score is selected; in the second iteration, j=2, the potential source biomolecule with the second “highest” biomolecule score is selected; etc. Another potential source biomolecule for comparison to the filter biomolecule is selected in step 5300. The selection 5300 is based primarily on the biomolecule score selected in 5100. That is, in step 5300, any potential source biomolecule with a selected biomolecule score lower than that of the filter biomolecule may be selected. A matched biomolecule fragment of the filter biomolecule is then selected, step 5400, for comparison to the matched biomolecule fragments of the biomolecule selected in step 5300.

The precise matched biomolecule fragment of the filter biomolecule selected is determined based on, for example, the closeness of the match to the corresponding mass signal, the linewidth associated with the matched mass signal, and/or investigator convenience. In one embodiment, only matched biomolecule fragments of the filter biomolecule that satisfy certain criteria of match closeness and/or detection likelihood are selected (“YES” to test 5410). In one embodiment, only matched filter biomolecule fragments that matched the corresponding mass signal within a select mass tolerance are selected (“YES” to test 5420). Accordingly, matched filter biomolecule fragments that do not satisfy this match closeness criterion (“NO” to test 5425) are not selected for comparison, result 5409. In one embodiment, a relative mass tolerance in the range from about 5 ppm to about 300 ppm is selected. In another embodiment, a relative mass tolerance in the range from about 25 ppm to about 100 ppm is selected. In another embodiment, a relative mass tolerance of 50 ppm is selected. However, it is to be understood that the specific value of the mass tolerance is not critical to the present invention, rather the mass tolerance is selected such that the mass signal-biomolecule fragment matches are statistically significant within a desired confidence interval. The confidence interval is determined based on the congestion of the mass spectrum, the resolution of the mass spectrum, accuracy of the biomolecule fragment masses in the list, and/or investigator convenience.

In another embodiment, only matched filter biomolecule fragments with an associated biomolecule fragment detection parameter above or below a cut off value (“YES” to test 5430) are selected. Accordingly, only matched filter biomolecule fragments with an associated biomolecule fragment detection parameter above the cut off value are selected when the detection probability or likelihood increases with increasing biomolecule fragment detection parameter and only those below the cut off value are selected when the detection probability or likelihood increases with decreasing biomolecule fragment detection parameter. It is preferred that the cut off value is selected such that biomolecule fragment detection parameters which do not provide a statistically significant contribution to the biomolecule score of the filter biomolecule are excluded from comparison, test 5440. In this manner, biomolecule fragments which make a statistically insignificant contribution to a filter biomolecule's biomolecule score do not result in the overall attenuation, via action 5441, of the biomolecule scores of biomolecules for which the biomolecule fragment contributes significantly to the biomolecule score. Matched filter biomolecule fragments that do not satisfy this detection probability criterion (“NO” to test 5435) are not selected for comparison, result 5409. In one embodiment, where the biomolecules of interest are proteins and the biomolecule fragment comprise peptides, one is selected as the minimum value for the biomolecule fragment detection parameter, where the detection probability or likelihood increases with increasing biomolecule fragment detection parameter and is determined substantially according to FIG. 4 and accompanying text.

Selected biomolecule fragments of the filter biomolecule, result 5401, are compared, test 5440, to the matched biomolecule fragments of the potential source biomolecule of step 5300. If the select filter biomolecule fragment is also a matched biomolecule fragment of the potential source biomolecule (“YES” to test 5440), then the biomolecule score associated with the matched biomolecule fragment of the potential source biomolecule of step 5300 is attenuated, action 5441, and the biomolecule score of that potential source biomolecule recalculated. The precise degree of attenuation is determined based on, for example, the relative biomolecule scores of the filter biomolecule and the potential source biomolecule of step 5300, the relative biomolecule fragment scores of the compared filter biomolecule fragment and the potential source biomolecule, the nature of the sample, and/or investigator convenience. In one embodiment, the biomolecule fragment score is attenuated by dividing by a number in the range from about 2 to 100. In another embodiment, the biomolecule fragment score is an attenuated by dividing by a number in the range from about 10 to 1000. In another embodiment, the biomolecule fragment score is attenuated by dividing by about 50. It is to be understood that the biomolecule fragment score is attenuated where the biomolecule fragment score increases with increasing significance of the mass signal-biomolecule fragment match, whereas the biomolecule fragment score is augmented where the biomolecule fragment score decreases with increasing significance of the mass signal-biomolecule fragment match.

Selection and comparison 5400 is repeated 5450 for the other matched biomolecule fragments of the filter biomolecule and for other select potential source biomolecules 5475. Further, steps 5200, 5300 and 5400 are repeated 5500 for a selected number of iterations. The precise number of iterations is based on, for example, the desired number of potential source biomolecules to be identified, the number of potential source biomolecules with a biomolecule score above or below a cutoff value, and/or investigator convenience.

In describing the determination of whether a biomolecule is likely present in or absent from a sample, reference will generally be made only to biomolecule scores; however, it is to be understood that such reference includes weighted biomolecule scores. Further, in the following discussion of determining whether a biomolecule is likely present or absent from a sample reference is made to the “highest” biomolecule score, or weighted biomolecule score. In the interest of a concise discussion, the phrase “highest” protein score, or weighted protein score, is meant to refer to the score that corresponds to the highest likelihood that the potential source biomolecule is present in the sample. Likewise, in the following discussion reference is made to the “highest” biomolecule fragment score and is meant to refer to the biomolecule fragment score that corresponds to the mass signal-biomolecule fragment match with the greatest significance. However, as discussed above, it is to be understood that it is not central to the present invention whether an increasing, decreasing, positive or negative biomolecule fragment score corresponds to a greater significance for a match or whether an increasing, decreasing, positive or negative biomolecule score corresponds to an increasing likelihood that the potential source biomolecule is present.

The likelihood of the presence or absence of a biomolecule(s) in a sample is determined from the biomolecule scores of the potential source biomolecules. This likelihood may be determined by comparison of biomolecule scores calculated according to a single formulation, e.g., such as determined via step 4100 of FIG. 5 without exclusion of select biomolecule fragments scores (“NO” to test 4120). Alternatively, the likelihood that a biomolecule is present or absent is determined by cross comparison of biomolecule scores calculated according to at least two formulations, e.g., such as the formulation of step 4100 and that of step 4600 in FIG. 5.

In certain instances an investigator wishes to determine whether a single biomolecule is likely present in or absent from the sample. In other instances, an investigator wishes to determine what biomolecules are likely present in a sample. In either instance, an investigator wishes to unambiguously determine whether a biomolecule is present or absent. In reality, however, different investigators have different criteria as to what constitutes an unambiguous determination. It is to be understood, however, that with a large mass list several mass signal biomolecule fragment matches may occur even if the associated biomolecule is not present in the sample. Accordingly, it is to be understood that a biomolecule score provides a likelihood of whether a biomolecule is present in or absent from a sample. In the present invention, several methods based on the biomolecule scores provided by the methods herein are used to assess the reliability of such a determination and provide a measure of the likelihood that a biomolecule is present in or absent from the sample.

In one embodiment, the number of matched biomolecule fragments of a potential source biomolecule are summed to determine a “biomolecule fragment count” for the potential source biomolecule. If this biomolecule fragment count is lower than a minimum number the potential source biomolecule is considered likely absent from the sample. The precise value of this minimum number is determined based on, for example, the size of the biomolecule, the suspected concentration of the biomolecule, the number of sites at which the biomolecule is readily fragmented, and/or investigator convenience. For example, a high value is preferred where the potential source biomolecule possess many amino acid bonds which are readily cleaved by the digest employed and a significant number of the resulting biomolecule fragments are known or predicted to have a high likelihood of detection.

In another embodiment, the number of matched biomolecule fragments of a potential source biomolecule that correspond to the N most intense mass signal intensities are summed to determine an “intense biomolecule fragment count” for the potential source biomolecule. In one embodiment, N is 100 and only matched biomolecule fragments that correspond to one of the 100 most intense mass signal intensities are counted. However, the precise value of N is not critical to the present invention; but rather, N is chosen based on factors such as the overall number of mass signals, the known or suspected concentration of the biomolecule(s) of interest, the congestion of the mass spectrum, and/or investigator convenience. If this intense biomolecule fragment count is lower than a minimum number the potential source biomolecule is considered likely absent from the sample. The precise value of this minimum number is also determined based on, for example, the size of the biomolecule, the suspected concentration of the biomolecule, the number of sites at which the biomolecule is readily fragmented, and/or investigator convenience. For example, a low value is preferred when the investigator is interested in establishing the absence of very minor components in a sample.

In one embodiment, the minimum biomolecule score which indicates a reliable determination that a biomolecule is likely present is determined by offsetting the entire mass scale of the mass spectrum. In this embodiment, the entire mass scale is shifted by a significant number of mass units, such as 3 amu. As a result, with a mass tolerance value of, for example 100 ppm or less, selected in general step 2000, no subsequent mass signal-biomolecule fragment matches should be statistically significant. Accordingly, where biomolecule score increases with presence likelihood, the maximum biomolecule score obtained by such offsetting represents the biomolecule score below which biomolecules cannot reliably be said to be likely present in the sample. In another embodiment, the biomolecule score calculated in the offset procedure excludes the highest biomolecule fragment score, or scores, from the calculation of the biomolecule score (“YES” to test 4120). This exclusion of the highest biomolecule fragment score, or scores, from the biomolecule score greatly reduces the possibility that a single fortuitous match to a mass signal with high intensity and/or with an associated biomolecule fragment with a high biomolecule fragment detection parameter will dominate the biomolecule score; and as a result, erroneously indicate the likely presence of certain biomolecules which are in fact absent from the sample. In another embodiment, a series of mass scale offsets are performed and the average biomolecule score obtained in the series of offsets is considered the minimum reliable biomolecule score. To further enhance the dependability of the minimum reliable biomolecule score, the biomolecule scores are also preferably weighted by the relative biomolecule fragment match count (“YES” to test 4600), in addition to excluding the highest biomolecule fragment score, or scores, from the biomolecule score determination.

Typically, the potential source biomolecule with the highest biomolecule score is present in the sample, assuming the database is sufficiently complete. The reliability of the determination process can be assessed by comparing biomolecule scores determined by other formulations of biomolecule scores described in general step 4000. In general, the reliability of the determination that the potential source biomolecule with the highest biomolecule score is present in the sample is best assessed by a biomolecule score which excludes the highest biomolecule fragment score, or scores, and/or the change in the biomolecule score upon weighting by the relative biomolecule match count. If the biomolecule score which excludes biomolecule fragment scores, and/or the biomolecule score weighted by the relative biomolecule match count, do not substantially change the ranking by biomolecule score of that potential source biomolecule relative to other potential source biomolecules, the determination that that potential source biomolecule is likely present in the sample increases in reliability.

In addition, the assessment of the reliability of a determination that a biomolecule is likely present in or absent from a sample is guided by the following additional principles which employ the biomolecule scores and/or weighted biomolecule scores of the present invention. In general, when a question exists as to whether a potential source biomolecule is present in the sample, and it is suspected that the biomolecule score thereof reflects a fortuitous match to a mass signal, the change in the biomolecule score of that potential source biomolecule, when the highest biomolecule fragment score, or scores, are excluded from the determination of the biomolecule score, with respect to the biomolecule scores of other potential source biomolecules so determined, is used to answer the question. For example, and ease of discussion, let the potential source biomolecule under question be referred to as A and another potential source biomolecule be referred to as B. If the biomolecule score of A becomes substantially lower than the biomolecule score of B then A is considered to have substantially decreased its ranking with respect to B. If A substantially decreases in ranking relative to B upon exclusion of the highest biomolecule fragment score(s), then the likelihood that B is present in the sample is considered significantly greater than the likelihood that A is present. Conversely, if the biomolecule score of A becomes substantially greater than that of B then A is considered to have substantially increased its ranking with respect to B. If A substantially increases in ranking relative to B upon exclusion of the highest biomolecule fragment score(s), then the likelihood that A is present is considered significantly greater than the likelihood that B is present.

In general, when there is a question regarding whether a potential source biomolecule that is known or predicted to generate relatively few biomolecule fragments upon digestion and/or fragmentation that have a reasonable likelihood of detection is present in the sample, the change in the biomolecule score of that potential source biomolecule, when weighted by the relative biomolecule detection parameter and/or the relative biomolecule match count, with respect to other potential source biomolecules so weighted, is used to answer the question. If such a potential source biomolecule is seen to substantially increase its ranking with respect to other potential source biomolecules based upon a the biomolecule score so weighted, that biomolecule is considered likely present in the sample.

In general, where there is a question whether a potential source biomolecule known or predicted to generate a relatively large number of biomolecule fragments upon digestion and/or fragmentation that have a reasonable likelihood of detection is present in the sample, the change in the biomolecule score of that potential source biomolecule upon weighting by the relative biomolecule intensity relative to the change in the biomolecule scores of the other potential source biomolecules so weighted, is used to answer the question. If such a potential source biomolecule is seen to significantly increase its ranking with respect to other potential source biomolecules based on comparison of the relative biomolecule intensity weighted biomolecule scores, that potential source biomolecule is considered likely present in the sample.

Where a question exists as to whether a potential source biomolecule is present in the sample and that potential source biomolecule is known or suspected to generate biomolecule fragment mass signals with high resolution in the mass spectrometry technique employed, such as when the biomolecule is present in high concentration, the change in the biomolecule score of that potential source biomolecule upon weighting by the biomolecule mass error relative to the change in the biomolecule scores of other potential source biomolecules so weighted is used to answer the question. If such a potential source biomolecule is seen to significantly increase its ranking with respect to other potential source biomolecules based on comparison of the biomolecule mass error weighted biomolecule scores that potential source biomolecule is considered likely present in the sample.

Finally, where a question exists as to whether a potential source biomolecule is present in a sample, and it is suspected that that potential source biomolecule is actually not present in the sample but nevertheless possesses a non-insignificant biomolecule score, the change in the biomolecule score of that potential source biomolecule upon weighting by the intensity weighted biomolecule mass error relative to the change in the biomolecule scores of other potential source biomolecules so weighted is used to answer the question. If such a potential source biomolecule is seen to significantly decrease its ranking with respect to other potential biomolecules based on comparison of the intensity weighted biomolecule mass error weighted biomolecule score, that potential biomolecule is considered not present in the sample compared to those potential source biomolecules of greater rank.

A special case of biomolecule presence determination occurs when two homologous, or identical sequence, biomolecules are present in the list to which mass signals and the mass spectrum are compared. The present invention generally determines a biomolecule score for one such biomolecule that is substantially higher than that of the other. Accordingly, it is preferred that potential source biomolecules be sorted and compared by biomolecule fragment sequence to determine whether homologous biomolecules may be present in the sample using substantially the scheme described in FIG. 6. When the masses corresponding to the potential source biomolecule with the highest biomolecule score are removed from consideration, the biomolecule scores of the lower ranking homologues are recalculated and typically are drastically reduced, because all or nearly all of the biomolecule fragments have already been accounted for by identical biomolecule fragments in the highest ranking homologue. In some cases, a sufficient number of distinct biomolecule fragments from a lower ranking homologue remain matched to mass signals not accounted for by the highest homologue to indicate that both homologous forms are actually present.

In another embodiment, to determine whether the identification of a potential source biomolecule is likely present in a sample is correct, is to consider more than one biomolecule score, weighted biomolecule score, or biomolecule score weighting parameter. Correctly identified biomolecules typically have the highest biomolecule score, as well as one of the highest weighted biomolecule scores and/or biomolecule score weighting parameters. For example, correctly identified biomolecules typically have one of the highest relative biomolecule intensity values, as well as one of the highest biomolecule fragment counts, and as well as one of the lowest intensity-weighted biomolecule mass errors. Because of this, potential source biomolecules with high biomolecule scores are less confidently identified if they have relative biomolecule intensity values below 20%, or which account for <5% of the intensity that cannot be attributed to higher ranking potential source biomolecules, or have a substantially higher intensity weighted biomolecule mass errors than higher ranking potential source biomolecules.

The present invention further provides a method to determine which mass signals of a sample's mass spectrum should be analyzed by a MS-MS technique to confirm or reject determinations of biomolecules and/or biomolecule fragments as likely present in or absent from the sample. This method uses the biomolecule fragment scores and/or biomolecule scores of the present invention to determine whether a mass signal should be subjected to MS-MS analysis; and thereby explicitly takes into account the likelihood of detecting a biomolecule fragment as a mass signal of the mass spectrum. In this embodiment, a mass spectrum of the resulting biomolecule fragments is obtained that produces a first spectrum of mass signals which primarily correspond to the biomolecule fragments that make up the biomolecule(s) present in the sample. The signal masses are compared to biomolecule fragment masses in a biomolecule fragment signal list that contains the biomolecule fragments known or predicted to be generated by the digestion and/or fragmentation of a given biomolecule. When a signal mass matches a biomolecule fragment mass, any biomolecule associated with that biomolecule fragment in the list is considered a potential source biomolecule for the corresponding mass signal. A biomolecule score is then assigned to the potential source biomolecule(s). The biomolecule fragment scores and/or biomolecule scores are used to determine whether a mass signal in the first mass spectrum is subjected to MS-MS analyses to confirm or reject a determination that a mass signal(s) in the first mass spectrum might correspond to a specific biomolecule fragment and/or biomolecule.

In one embodiment, mass signals matched to biomolecule fragment(s) with the high(est) biomolecule fragment scores(s) are subjected are subjected to MS-MS analysis to confirm or reject the mass signal-biomolecule fragment match. In another embodiment, mass signals that correspond a potential source biomolecule with a high likelihood of being present, as indicated by the biomolecule score, are subjected to MS-MS analysis to confirm or reject the determination that the biomolecule is likely present in the sample. In the above two embodiments, the high(est) scoring biomolecule fragment(s) or biomolecule(s) are chosen for further analysis because such high scores typically arise from the biomolecule(s) most prevalent in the sample. Accordingly, the above two embodiments are preferable when the biomolecule suspected to be present in sample and/or the biomolecule of interest is the most prevalent biomolecule(s).

In another embodiment, the most intense mass signal(s) that does not match a biomolecule fragment in any list used in step 2000, i.e., an “unexplained” mass signal, is subjected to MS-MS analysis to provide further information and assist in determining a source molecule(s) or potential source biomolecule(s) for the mass signal(s). In this embodiment, the “unexplained” mass signal corresponds to an undetermined likelihood of a biomolecule fragment being detected, because no biomolecule fragment score is determined for an unmatched mass signal.

In another embodiment, mass signals that correspond only to a potential source biomolecule with a low or very low likelihood of being present are subjected to MS-MS analysis to confirm or reject the determination that the biomolecule might be present in the sample. This embodiment is preferable when the biomolecule(s) suspected to be present in the sample and/or the biomolecule(s) of interest are present in low concentration yet comprise biomolecule fragment(s) with high likelihoods of being detected.

In another embodiment, MS-MS analysis is used to distinguish between multiple potential source biomolecules which possess comparable biomolecule scores to confirm or reject if any or all of the biomolecules are present in or absent from the sample. In another embodiment mass signals that correspond to two or more potential source biomolecules with comparable biomolecule scores are subjected to MS-MS analysis to confirm or reject the source biomolecule(s) of the mass signals.

To confirm or reject a determination that a mass signal(s) in the first mass spectrum correspond to a specific biomolecule fragment and/or biomolecule, a second mass spectrum is obtained of the mass signal that the program has determined should be subjected to MS-MS analysis. In this manner, a mass fingerprint of the mass signal in the first mass spectrum is obtained that can serve to confirm or reject the determination that this mass signal(s) corresponds to a specific biomolecule fragment or arises from a given biomolecule. Any suitable method of determining the source molecule(s) of the first mass spectrum mass signal from the second mass spectrum can be used, including those of the present invention.

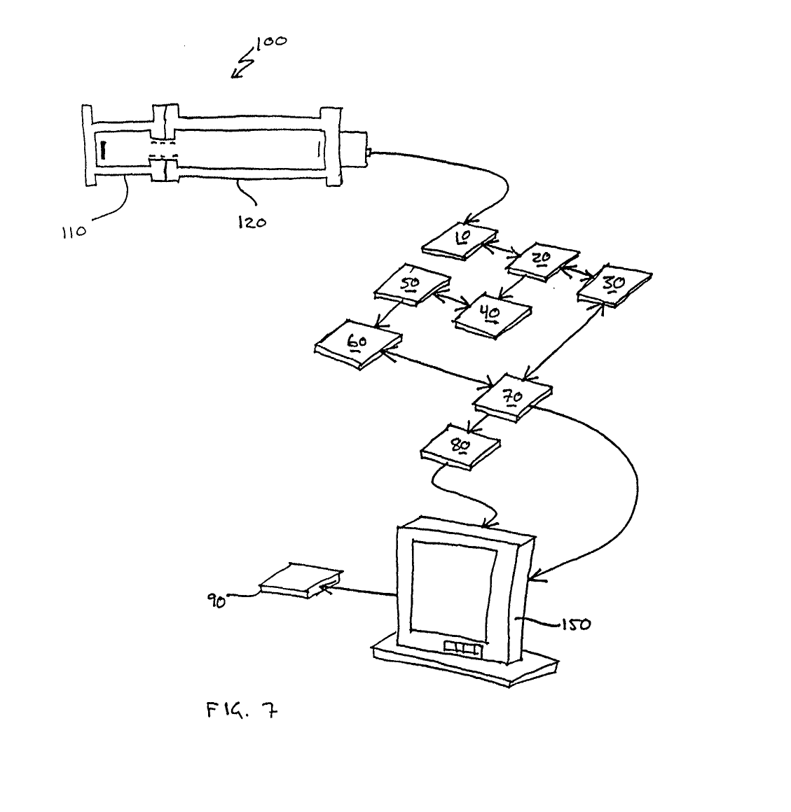

The present invention further provides an apparatus for determining the likelihood of the presence or absence of a protein in a biological sample using the methods of the present invention. Referring to FIG. 7, in one embodiment, the apparatus comprises a mass spectrometry instrument 100 that obtains a mass spectrum of a sample. The mass spectrometry instrument 100 is comprised of a ionization instrument 110 and a ion separation instrument 120 which may function as a “biomolecule fragment separation apparatus.” The mass spectrometry instrument 100 obtains a mass spectrum as follows. A portion of the digestion and/or fragmentation product of the sample, containing biomolecule segments and biomolecule fragments of sample biomolecules, is ionized by the ionization instrument 110. In one embodiment, the ionization instrument comprises a MALDI instrument; in another, it comprises an electrospray ionization instrument. However, the specific ionization instrument, or method of ionization, is not crucial to the present invention; any suitable instrument for, or method of, ionization of a biomolecule is contemplated by the present invention. The resulting ions are transported to the ion separation instrument 120 by any suitable method, such as by electrophoresis, gas phase chromatography, liquid phase chromatography, electrostatic elements, electromagnetic elements, or radio-frequency elements. In one embodiment, the ion separation instrument comprises a TOF mass spectrometer; in another, it comprises a quadrupole mass spectrometer. In still another embodiment, the ion separation instrument comprises a chromatography system. However, the specific ion separation instrument employed is also not crucial; any suitable instrument is contemplated by the present invention. The ion separation instrument 120 separates ions as a function of ion mass and the ion signal intensity as a function of ion mass is measured. The ion signal intensity as a function of ion mass constitutes the raw data of which the mass spectrum is comprised while a single ion signal for an ion mass comprises a “mass signal.”

Referring again to FIG. 7, the mass spectrometry instrument 100 is in communication with a first memory element 10 that stores the mass spectrum obtained by the mass spectrometry instrument 100. The mass spectrum obtained by the mass spectrometry instrument 100 is transmitted to the first memory element 10. The first memory element 10 is accessed by a comparator, stored in a second memory element 20, which compares data corresponding to mass signals of the mass spectrum to a list stored in a third memory element 30 that stores the masses of biomolecule fragments known or predicted to be generated by the digestion and/or fragmentation of select biomolecules. The comparator accesses the third memory element 30, searches the list stored thereon and compares signal masses to biomolecule fragment masses in the list according to the methods disclosed herein to determine if they match. If the comparator determines that a signal mass matches a biomolecule fragment mass, the mass signal-biomolecule fragment match is transmitted to and stored in a fourth memory element 40.

A fifth memory element 50 containing a weight generator accesses the fourth memory element 40 to determine a biomolecule fragment score for a mass signal-biomolecule fragment match according to the methods disclosed herein. The biomolecule fragment scores determined by the weight generator are transmitted to and stored in a sixth memory element 60. A combination generator contained in a seventh memory element 70 accesses the sixth memory element 60 and the third memory element 30 and combines the biomolecule fragment scores stored in the sixth memory element 60 of an associated select biomolecule stored in the third memory element 30 to generate a biomolecule score for that biomolecule according to the methods disclosed herein. In one embodiment, the seventh memory element 70 containing the combination generator is in communication with an output device 150 that enables an investigator to compare biomolecule scores and thereby determine the likelihood of the presence or absence of a biomolecule(s) in the sample. In another embodiment, the biomolecule scores determined by the combination generator are transmitted and stored in an eighth memory element 80 which in turn is in communication with an output device 150 that enables an investigator to compare biomolecule scores and thereby determine the likelihood of the presence or absence of a biomolecule(s) in the sample.

In one embodiment, the output device 150 produces a human readable display, for example, such as that produced by a printer or computer screen. However, it is not crucial the present invention whether the output device produces either a human readable and/or machine readable only output. For example, the output device may produce machine readable only data which is transmitted to and stored in a ninth memory element 90. In this embodiment, the investigator might determine the likelihood of the presence or absence of a biomolecule in a sample by means of an algorithm which evaluates the biomolecule scores and determines the likelihood of the presence or absence of a protein based on the rules of the algorithm.

The memory elements described herein may be discreet memory elements that receive data and are accessed by the comparator, a weight generator, and/or combination generator. Alternately, the memory elements may refer to a portion of random access memory which is set aside to store the data transmitted thereto. In some embodiments, the functionality described above may be implemented as software on a general purpose computer. The computer may be separate from, detachable from, or integrated into the mass spectrometry instrument 100. In addition, such a program may set aside portions of a computer's random access memory to provide the comparator, the weight generator, and the combination generator program logic that affect comparisons between and the operations with and on the data stored in the memory elements. In such an embodiment, the program may be written in any one of a number of high-level languages, such as FORTRAN, PASCAL, C, C++, or BASIC. Further, the program may be written in a script, macro, or functionality embedded in commercially available software, such as EXCEL, Data Explorer (Applied Biosystems) or VISUAL BASIC. Additionally, the software could be implemented in an assembly language directed to a microprocessor resident on a computer. For example, the software could be implemented in Intel 80×86 assembly language if it were configured to run on an IBM PC or PC clone. The software may be embedded on an article of manufacture including, but not limited to, “computer-readable program means” such as a floppy disk, a hard disk, an optical disk, a magnetic tape, a PROM, an EPROM, or CD-ROM.

The present invention is illustrated further by the following non-limiting examples.

### EXAMPLES

All experiments were performed on a Applied Biosystems Voyager DE-PRO or DE-STR, using Voyager 5.0 Control software.

### Example 1

### Sample Analysis Employing a Trypsin Digest of Sample Proteins and MALDI-TOF Mass Spectrometry Technique

The sample was prepared following a protocol adapted from Frangioni and Neal, Anal. Biochem. 21079-187 (1993). The sample, E. coli strain DH5 (IL), was grown in LB broth at 37° C. in four 500 mL bottles (250 mL of broth each). Cells were washed in 10 mM MOPS pH 7.2, and lysed in 40 mL of 4M urea/5 mM MOPS pH 7.2 containing 100 ug/ml lysozyme to extract the proteins from the cells. The lysate containing the extracted proteins was then incubated for 15 min. at 0° C., stock reagent solutions were added to make a final concentration of 5 mM dithiothreitol and 1 mM PMSF, and frozen at −20° C. in aliquots. 50 ul aliquots were mixed with a 2D gel electrophoresis sample buffer containing urea and then absorbed into an Immobiline strip, pH range 4.0 to 9.0, and focused for 24 h at 3000V using a Pharmacia Multiphor2. The rehydrated gel was loaded onto a Laemmli SDS gel, soaked in Laemmli sample buffer, and subjected to electrophoresis for 3 hours. The gel was washed once in water, stained with colloidal Coomassie (Gel code blue, Pierce) for 2 hours, and then destained in water. Gel spots were excised using a truncated 20 μl micropipet tip and either rinsed onto the top of a 96 well filter plate or submitted to a Genomics Solution ProGest digestor. The 96 well filter plate was placed onto the deck of a Genesis RSP 150 robot (Tecan, Rapperswill, Switzerland) equipped with a vacuum manifold and a set of eight 0.5 ml syringe pumps. 200 μl of 50% acetonitrile/20 mM ammonium bicarbonate was added to each well, incubated for 15 min., then drawn away 2 to 4 times using the vacuum manifold. Subsequently, 100 μl of acetonitrile was added and after 5 min. the solution was drawn away. 100 μl of TPCK-treated bovine trypsin (Sigma), at 2 μg/ml in 10 mM ammonium bicarbonate was added to digest the proteins. After 5 min., the excess liquid was drawn away, 20 μl of 5 mM ammonium bicarbonate was added, and the plate was sealed and incubated at 37° C. for 18 hours. The peptides were then extracted by adding 50 μl of 50% acetonitrile/15% TFA, and drawn into a polypropylene microtiter plate catch plate using the vacuum manifold or, alternatively, by centrifuging at low speed in a SpeedVac. The peptide extracts were then dried in the SpeedVac.

The resulting dried peptide samples were dissolved in 1.5-10 μl of 50% acetonitrile/0.1% TFA, and 0.5 μl of the resultant solution was spotted onto a MALDI sample holder and 0.5 μl of matrix solution containing 10 mg/ml α-cyano-4-hydroxy-trans-cinnamic acid dissolved in 50% acetonitrile was added to each spot. The MALDI sample holder was then placed in a Applied Biosystems Voyager DE-PRO and a mass spectrum of the sample obtained. The peptide samples were ionized using a laser irradiance of between 1400 and 2000 and 100-800 mass spectra were summed to obtain a mass spectrum.

Data Explorer (Applied Biosystems) software was used to identify peaks, i.e. mass signals, of the mass spectrum. The mass spectrum was then corrected for electronic noise, baseline and mass signal isotopic variants. The mass spectrum was then internally calibrated by either: (1) calibration masses used at the time of data acquisition, for example, bovine trypsin 2163.0570 and 1153.57; (2) manual calibration using mass signals previously identified; or (3) by the Applied Biosystems PS1 IntelliCal function. The data presented below were found to not be significantly affected by the calibration employed.

The six to ten most intense mass signals per 100 amu of the mass spectrum, from 800 amu to 3600 amu, were selected for comparison. This resulted in 50-270 mass signals being selected for comparison. The selected mass signals were compared to two lists. The first list contained commonly encountered contaminant proteins such as BSA, lysozyme, bovine trypsin and human keratins, namely, k1, k2, k9, and k10. The first list also contained two “pseudoproteins” the first pseudoprotein consisted of peptide and Coomassie blue (mass 832 amu) calibration standards; the second pseudoprotein consisted of contaminants commonly encountered, yet unidentified, with the mass spectrometry system when employed for biomolecule analysis. The second list contained E. coli proteins and was derived from SwissProt release 3.6, and contained over 275,000 predicted peptide signals for more than 4000 E. coli proteins. The selected mass signals were compared to both lists to determine which proteins were likely present in the sample. The data obtained for 218 E. coli proteins is summarized in Table 1. The data was obtained from a series of one and two dimensional gels on E. coli proteins digested with trypsin.

TABLE 1

| 1 | 2 | 3 | 4 | 5 | 6 | 7 |
|---|---|---|---|---|---|---|
| Match Criteria | num | Arg | Lys | X | p | Cys |
| match # > 4 | 218 | 55.6 | 9.6 | 3.2 | 22.1 | 1.8 |
| match # > 9 | 57 | 66.3 | 8.3 | 1.5 | 16.4 | 0.8 |
| match # > 14 | 20 | 65.7 | 6.6 | 0.9 | 19.4 | 0.3 |
| IBME < 20 | 197 | 57.8 | 9.5 | 2.8 | 21.8 | 1.8 |
| IBME < 10 | 120 | 62.1 | 8.0 | 2.8 | 20.5 | 1.3 |
| IBME < 5 | 23 | 72.1 | 6.2 | 2.1 | 13.2 | 2.0 |
| % I match > 10 | 87 | 64.5 | 8.9 | 2.8 | 17.8 | 1.2 |
| % I match > 20 | 41 | 67.7 | 7.2 | 0.9 | 18.7 | 0.3 |
| % I match > 30 | 26 | 71.2 | 8.8 | 0.7 | 14.3 | 0.3 |

Referring to Table 1, the columns list the average contribution to the total matched mass signal intensity for peptides as a function of peptide sequence and/or degree of peptide digestion. The rows of Table 1 list this data as obtained for a variety of matching criteria, such as mass tolerance or number of peptides matched. Column 1 notes the matching criteria of a given row. Column 2 shows the number of proteins in the second list satisfying the matching criteria of column 1.

Rows 1-3 contain data for the matching criteria of test 2540 where, respectively, greater than 4, greater than 9, and greater than 14, mass signal-biomolecule fragment matches were required. Rows 4-6 contain data for matching criteria where a maximum intensity weighted biomolecule mass error of, respectively, less than 20 ppm, less than 10 ppm, and less than 5 ppm, was required. Rows 7-9 contain data for matching criteria where the percentage of the biomolecule intensity matched was required to be, respectively, greater than 10%, greater than 20%, and greater than 30%.

Column 3 shows that the average contribution for fully digested Arg containing peptides is about 55 to 72% of the observed matched signal intensity. Regardless of whether stringency was increased by increasing the required number of matching peptides, by requiring a lower intensity weighted biomolecule mass error, or by requiring a higher percent of matched signal intensity, the percentage of intensity attributable to completely digested Arg containing peptides increased slightly, indicating that the more confident the identification, the more likely the intensity would be accounted for by Arg containing peptides. Column 4 shows that the average contribution for fully digested Lys containing peptides is about 6 to 10% of the observed matched signal intensity. Comparison of columns 3 and 4 shows that the ratio of Arg containing peptide intensity to Lys containing peptide intensity can vary from about 12 to 1 to approximately 5 to 1. As a result, in one embodiment, where the biomolecule fragment detection parameter increases with increasing probability of detection, fully digested Arg containing peptides are assigned a base biomolecule fragment detection parameter of ten (value A in FIG. 4B) whereas fully digested Lys containing peptides are assigned a base value of one (value B in FIG. 4B). The number of fully digested peptides containing arginine detected divided by the number of possible arginine-containing peptides was also calculated, and compared to the fraction of fully digested peptides containing lysine but not arginine. It was observed that the percentage of fully digested Arg containing peptides detected varied from about 67% to about 100% depending on the protein, with an average of roughly 82%. Similarly, it has been observed that the fraction of fully digested Lys containing peptides observed can vary from about 20% to about 62%, with an average of roughly 42%. Accordingly, the ratio of Arg containing peptide fraction observed to Lys containing peptide fraction observed can vary from about 5 to 1 to approximately 3 to 2. In one embodiment, the base value of the biomolecule fragment detection parameter takes into consideration both the relative intensity and relative fraction observed of the biomolecule fragments produced by the fragmentation process used, e.g. Arg and Lys containing peptides when a trypsin digest is used. As a result, in one embodiment, where the biomolecule fragment detection parameter increases with increasing probability of detection, the ratios of the base values of fully digested Arg-containing peptides (value A in FIG. 4B) to the value for fully digested Lys containing peptides (value B in FIG. 4B) is in the range from about 10:1 to 3:2. In one embodiment, the ratio of value A to B is about 10:1. In another embodiment, the ratio of value A to B is about 8:3. However, it should be realized that the exact numerical value of the base biomolecule fragment detection parameter is not crucial to the present invention, and also has little impact on the results It is to be understood that the base numerical value need only roughly reflect the relative intensity and/or relative fraction observed between fully digested Arg containing and Lys containing peptides.

Table 1 further shows that the average contribution to the total matched mass signal intensity also varies with biomolecule fragment composition. Column 5 lists the observed contribution of missed cleavage peptides not containing the sequences listed in FIG. 4C, steps 3230-3248 and the accompanying text. Column 5 shows that such missed cleavage peptides contribute about 0.7 to 3.2% of the total matched mass signal intensity. Accordingly, the data indicates that the base value of the biomolecule fragment detection parameters of missed cleavage peptides should be adjusted to reflect this decreased detection likelihood. To reflect this result, where the biomolecule fragment detection parameter increases with increasing likelihood of detection, the base value for missed cleavage peptides is reduced.

Column 6 lists the observed contribution of missed cleavage peptides containing the sequences listed in FIG. 4C, steps 3230-3248 and the accompanying text. Column 6 shows that missed cleavage peptides with such sequences have a signal intensity more comparable to fully digested peptides. Accordingly, the data indicate that the effect of a missed cleavage for missed cleavage peptides with such sequences should be adjusted to reflect this result. Consequently, where the biomolecule fragment detection parameter increases with increasing likelihood of detection, the base value for missed cleavage peptides with such sequences is reduced less than that of missed cleavage peptides without these sequences.

Referring to Table 1, column 7, data is shown for the contribution of unalkylated Cys-containing peptides to the total matched mass signal intensity. Column 7 indicates that unalkylated Cys-containing peptides contribute about 0.3 to 2% of the total matched mass signal intensity. For many proteins, no Cys-containing peptides were detected even though many such peptides were predicted.

### Example 2

### Use of the Biomolecule Fragment Detection Parameter for Methionine

The level of oxidation of methionine is found to vary between different experiments. The data in Table 2 show the results from three representative experiments, denoted experiments 1d-1, 1d-2, and 2d. The results for experiments 1d-1, 1d-2, and 2d appear, respectively, in rows 1, 2, and 3. In the first two experiments, 1d-1 and 1d-3, proteins were isolated from slices from an SDS gel, whereas in the third experiment, proteins were isolated from a 2d gel. Column 2 list the number of spectra that were analyzed in the experiment, while column 3 lists the number of proteins that were identified. Column 4 list the percentage of the intensity due to methionine-containing peptides that was accounted for by peptides containing oxidized methionine. It can be seen that in Experiment 1d-1, only about 20% of the methionine in the matched peptides was oxidized, whereas in 1d-2, about half of the methionine was oxidized. In the 2d gel experiment, almost all of the methionine was oxidized. Accordingly, the present invention takes these findings into consideration by modifying the biomolecule fragment detection parameter for oxidized Met-containing peptides based on the observed degree of oxidation. This would cause the biomolecule scores for those potential source proteins whose peptides have the observed degree of methionine oxidation to increase. Typically, this gives these potential source proteins a sorting advantage compared to those whose peptides are outliers with respect to methionine oxidation.

TABLE 2

| 1 | 2 | 3 | 4 |
|---|---|---|---|
| Experiment | spectra | proteins | % Mo |
| 1d-1 | 84 | 125 | 20.2 |
| 1d-2 | 76 | 337 | 54.7 |
| 2d | 86 | 69 | 96.1 |

It is to be realized that the exact numerical values of the base biomolecule fragment detection parameters, and the modifications thereto, are not crucial to the present invention. For example, the absolute numerical values may be larger or smaller and the modifications to the base value for the effects of biomolecule fragment sequence and/or neighboring amino acids can be augmented or diminished. While the determination of biomolecule fragment detection parameters has been particularly shown and described with respect to an analysis employing a trypsin digest and a MALDI-TOF mass spectrometry technique, it should be understood by those of ordinary skill in the art that the principles illustrated herein may be applied to any digest-mass spectrometry technique combination. Further, it should be understood by those skilled in the art that while E. coli peptides were analyzed to determine the relative intensity relationship of peptides as a function of sequence and neighboring amino acids, that the biomolecule fragment detection parameters determined therefrom may be employed to determine the presence or absence of protein(s) in other biological samples

### Example 3

### Identification of a Minor Protein Component in a Mixture

A standard mixing experiment was performed starting from solution digests of E. coli b-galactosidase (gal; MW 116358) and rabbit phosphorylase (phos; MW 97098) to test the ability of the present invention to identify minor protein components in a mixture of proteins. Samples were prepared and a mass spectrum obtained substantially as described above and over 100 mass signals were used for comparison.

Protein mixtures were prepared with the following ratios of gal to phos: 10:1; 20:1; 1:10; and 1:30. A mass spectrum was obtained for each mixture and approximately 150 of the most intense mass signals of each spectrum were selected for comparison to both a list of E. coli proteins and a list containing rabbit phosphorylase. Prior to comparison, the mass spectrums were calibrated, corrected for electronic noise, baseline, and isotopic variants, and mass signals attributable to common contaminants were removed. The select mass signals were subsequently analyzed according to the methods described herein to determine the proteins present in the mixture. Biomolecule scores were determined substantially in accordance with equation 1 or 2 with a value of 2 ppm set for ppmₘᵢₙ. The following parameters were used to identify potential source biomolecules in general step 2000: (1) a mass tolerance value of 50 ppm was used in step 2310; (2) a minimum biomolecule fragment match number of 3 was used in step 2530 (i.e., “YES” to tests 2510 and 2520); (3) the mass signal intensity was required to be one of the 70 most intense selected mass signals in step 2545 (i.e., “YES” to test 2540); and (4) a minimum biomolecule fragment detection parameter of 1 was required where the biomolecule fragment detection parameters were determined substantially in accordance with the method illustrated by FIGS. 4A-E and accompanying text.

The biomolecule fragment detection parameters were determined substantially in accord with FIG. 4B with “NO” to tests 3120 and 3140 chosen and base values of 8, 3, and 0.8 were chosen, respectively, for base values A, B and C. The biomolecule fragment detection parameters were determined substantially in accord with FIG. 4C except tests and actions 3240 to 3249 were not used. Values of 50, 50, 20, 20, 20, and 10 were chosen, respectively, for factors D1, M1, M2, M3, M4, and M5, and values of 0.9, 0.95, 0.9, 0.9, and 0.95 were chosen, respectively, for factors F1 to F5. The biomolecule fragment detection parameters were determined substantially in accord with FIG. 4D where values of 1, 10, and 50 were chosen, respectively, for factors D2 to D4. The biomolecule fragment detection parameters were determined substantially in accord with FIG. 4E where a value of 1 was chosen for factor S1.

Biomolecule scores, and weighted biomolecule scores, for the identified potential source biomolecules were then determined substantially in accordance with the methods illustrated by FIGS. 5, 6 and accompanying text. Duplicate mass signal matches were excluded (“YES” to tests 4110 and 4750) and protein scores were calculated without exclusion of select peptide scores (“NO” to test 4120) and with the exclusion of the highest biomolecule fragment score (“YES” to test 4120). The biomolecule scores were refined in accordance with the methods illustrated by FIG. 6 and accompanying text. The filter biomolecule was selected based on the biomolecule score determined by the formulation of step 4100 with duplicate matches excluded and the highest biomolecule fragment score excluded (“YES” to test 4110 and “YES” to test 4120). Biomolecule fragments of a filter biomolecule were only selected (“YES” to test 5410) for use as a filter biomolecule fragment if the mass error of the mass signal-biomolecule fragment match for that biomolecule fragment was less than or equal to 25 ppm (criteria for test 5425) and the biomolecule fragment detection parameter of that biomolecule was greater than or equal to 1 (criteria for test 5435), where the biomolecule fragment detection parameter was determined substantially in accordance with the method illustrated by FIGS. 4A-E and accompanying text as indicated above.

Referring to Table 3, a summary of the biomolecule scores and weighted biomolecule scores determined for the four protein mixtures are shown. The first column of Table 3 indicates the mixture for which the results were obtained. The second column indicates the potential source biomolecule and the third column the molecular weight of this biomolecule. The fourth column lists the number of biomolecule fragments (i.e., peptides) matched to the potential source biomolecule within a mass tolerance of 25 ppm, that had a biomolecule fragment detection parameter greater than 1, and which were not subsequently attenuated, i.e., “YES” to test 5440, whereas column 5 lists the number of biomolecule fragments matched to the potential source biomolecule within a mass tolerance of 50 ppm regardless of the biomolecule fragment detection parameter or matches to other biomolecules. Column 6 lists the relative biomolecule detection parameter (weight of step 4400), column 7 lists the normalized (or dimensionless) biomolecule score determined substantially according to equation (2) and by the formulation of step 4100 with duplicate matches exclude but all biomolecule fragment scores included (“YES” to test 4110 and “NO” to test 4120), column 8 lists the biomolecule score determined by the formulation of step 4100 with duplicate matches 10 excluded and the highest biomolecule fragment score excluded (“YES” to test 4110 and “YES” to test 4120). Finally, column 9 lists the relative biomolecule intensity (weight of step 4500) and column 10 lists the relative biomolecule mass error (weight of step 4700).

TABLE 3

| 1 | 2 | 3 | 4 | 5 | 6 | 7 | 8 | 9 | 10 |
|---|---|---|---|---|---|---|---|---|---|
| ratio | Protein | MW (amu) | m | mo | rbdp | biomol. score | score minus | % I | mass error |
| 10:1 G:P | beta galactosidase | 116358 | 34 | 39 | 49.8 | 787 | 578746 | 56.9 | 4.9 |
| 10:1 G:P | rabbit phosphorylase | 97098 | 15 | 20 | 23.4 | 27 | 9812 | 5.6 | 8.7 |
| 10:1 G:P | mobilization protein | 19550 | 3 | 5 | 14.0 | 5 | 962 | 1.1 | 3.4 |
| 20:1 G:P | beta galactosidase | 116358 | 33 | 38 | 52.2 | 889 | 48493 | 68.5 | 4.6 |
| 20:1 G:P | rabbit phosphorylase | 97098 | 9 | 13 | 15.5 | 4 | 107 | 2.0 | 15.7 |
| 20:1 G:P | finq protein | 39943 | 5 | 6 | 20.4 | 5 | 87 | 2.8 | 12.8 |
| 10:1 P:G | rabbit phosphorylase | 97098 | 31 | 40 | 51.9 | 747 | 280970 | 85.1 | 3.5 |
| 10:1 P:G | beta galactosidase | 116358 | 15 | 19 | 23.4 | 4 | 480 | 1.3 | 25.3 |
| 10:1 P:G | hypothetical 32 5 kd protein | 32527 | 1 | 4 | 11.9 | 3 | 214 | 2.7 | 30.0 |
| 30:1 P:G | rabbit phosphorylase | 97098 | 41 | 52 | 58.5 | 1080 | 64045 | 76.4 | 5.1 |
| 30:1 P:G | beta galactosidase | 116358 | 15 | 17 | 22.7 | 9 | 652 | 2.6 | 9.0 |
| 30:1 P:G | dna 3 methyladenine glycosidase | 31398 | 3 | 5 | 19.8 | 2 | 73 | 0.7 | 18.0 |

Referring to Table 3, the potential source biomolecules with the three highest biomolecule scores are listed. In each case, the first protein listed corresponds to the major component, while the second protein listed corresponds to the minor component. The potential source biomolecule with the third highest biomolecule score is also listed to illustrate the scores for a match to “background” mass signals. Thus, the methods of the present invention were able to identify correctly the minor component out of a list of more than 4000 possible proteins, even though the minor component could account for only 1.3% to 5.6% of the total intensity (see column 9). Note that the proteins were sorted according to the biomolecule score in column 8. It can be seen that in two cases, 10:1 G:P and 30:1 P:G, the score in column 8 for the minor component was substantially higher than the score for the background protein. In a third case (10:1 P:G), the minor component had a score about twice that of background, while in the fourth case (30:1 P:G), the correct minor component was barely higher than background.

Applying the general principles described above, the question of whether the minor component is likely present in, or absent from, the sample can be addressed as follows. As discussed above, when it is suspected that the biomolecule score of a potential source biomolecule reflects a fortuitous match to a mass signal, the change in the biomolecule score of that potential source biomolecule, when the highest biomolecule fragment score, or scores, are excluded from the determination of the biomolecule score, with respect to other potential source biomolecules, can be used to determine whether the biomolecule is likely present in the sample. As indicated by the data of Table 3, an investigator not knowing the proteins present in the sample analyzed might well suspect that the biomolecule scores of both the minor protein component and the “background” match are the result of a fortuitous match to a mass signal. Because the methods of the present invention calculate multiple parameters that correlate with correct identification of the biomolecule(s) likely present in a sample, the investigator can assess the reliability of a identification(s) by looking at how many of these parameters track in parallel. For the 10:1 G:P sample, the parameters in columns 3-9 for the minor component are all higher than for the third component, indicating a relatively robust identification. For the 20:1 G:P sample, however, only the parameters in columns 4, 5 and 8 are higher for the minor component, indicating a borderline identification. However, employing the methods of the present invention, biomolecule scores are determined which exclude the highest biomolecule fragment score. A comparison of columns 7 and 8 reveals that depending on how the biomolecule score is calculated, minor components can be identified. It is observed that for the minor protein component the biomolecule score with highest biomolecule fragment score excluded, column 8, increases relative to the same score for “background” match, and that the biomolecule score of the background match decreases relative to both the minor and major protein components. The gal to phos ratio of 20:1 the relative difference between the minor protein component (phos) and the background match (finq) biomolecule scores, however, is only about 20%, the phos biomolecule score changes from about 20% lower than that of finq to about 20% higher when the highest biomolecule fragment score is excluded. Accordingly, the swing in the relative difference between the finq and phos biomolecule scores is roughly 40%, tentatively indicating that phos is more likely present than finq. As a result, the methods of the present invention provide only a tentative indication that the minor protein component is present in the sample and that the “background” match is absent while still correctly identifying the major component as present in the sample. Without being held to theory, it is believed that this tentative identification occurs because it is much harder to identify small amounts of large proteins (or biomolecules) than smaller proteins (or biomolecules). This is believed to occur because the matching of only a few key peptides (i.e., biomolecule fragments) in a small protein causes the relative biomolecule detection parameter to increase significantly for small proteins, whereas no individual peptides are similarly crucial for the identification of larger proteins.

While the invention has been particularly shown and described with reference to specific embodiments, it should be understood by those skilled in the art that various changes in form and detail may be made therein without departing from the spirit and scope of the invention as defined by the appended claims.

What is claimed is:

1. A method of determining the likelihood of the presence of a biomolecule comprising the steps of:
providing at least one mass signal, wherein said mass signal has a mass signal intensity;
comparing said mass signal to a list of known biomolecule fragment signals to determine a potential source biomolecule of said mass signal, wherein said mass signal corresponds to at least one biomolecule fragment of said potential source biomolecule;
determining a biomolecule fragment score for said mass signal, wherein said biomolecule fragment score comprises a function of a detection likelihood for said mass signal which defines a biomolecule fragment detection parameter;
repeating the steps of comparing and determining a biomolecule fragment score as necessary for additional mass signals;
combining said biomolecule fragment scores of said mass signals that correspond to a known biomolecule fragment list for said potential source biomolecule, said combination defining a biomolecule score for said potential source biomolecule;
repeating the step of combining as necessary for additional potential source biomolecules; and
determining the likelihood of the presence or absence of said biomolecule based on said biomolecule score of said potential source biomolecule that corresponds to said biomolecule.

2. The method of claim 1 wherein the step of determining the likelihood of the presence or absence of said biomolecule further comprises the steps of:
selecting one of said at least one mass signal, which defines a selected mass signal;
comparing said biomolecule scores of at least two potential source biomolecules that correspond to said selected mass signal to determine said potential source biomolecule with a highest biomolecule score;
determining that said biomolecule is likely absent if said biomolecule score of the corresponding potential source biomolecule is lower than said highest biomolecule score; and
repeating the steps of selecting, comparing biomolecule scores and determining as necessary for additional selected mass signals.

3. The method of claim 1 further comprising the step of correcting said mass signal intensity for an isotopic variant of a biomolecule fragment which corresponds to said mass signal.

4. The method of claim 1 further comprising the step of calibrating a mass which corresponds to said mass signal.

5. The method of claim 1 further comprising the step of removing noise from said mass signal intensity.

6. The method of claim 1 further comprising the step of removing artificial background intensity from said mass signal intensity.

7. The method of claim 1 wherein said biomolecule fragment detection parameter is proportional to the detection efficiency of said biomolecule fragment that corresponds to said mass signal and a probability that said potential source biomolecule yields said biomolecule fragment as a result of a fragmentation process applied to said potential source biomolecule.

8. The method of claim 1 wherein the step of determining a biomolecule fragment score for said mass signal comprises the steps of:
determining a detection likelihood for said mass signal which defines said biomolecule fragment detection parameter;
determining a mass error for said mass signal from the relative difference between a mass which corresponds to said mass signal and a mass of a known biomolecule fragment which corresponds to said mass signal; and
determining said biomolecule fragment score from said mass signal intensity, said biomolecule fragment detection parameter, and said mass error for said mass signal.

9. The method of claim 8 wherein said biomolecule fragment score is proportional to said biomolecule fragment detection parameter and said mass signal intensity, and inversely proportional to said mass error for said mass signal.

10. The method of claim 1 wherein the step of combining said biomolecule fragment scores comprises excluding a selected biomolecule fragment score from said combination.

11. The method of claim 1 wherein the step of combining said biomolecule fragment scores further comprises the steps of:
determining a biomolecule fragment count of said potential source biomolecule by counting the number of said mass signals that correspond to said potential source biomolecule;
comparing said biomolecule fragment count to the number of said biomolecule fragments on a known biomolecule fragment list for said potential source biomolecule to determine a relative biomolecule match count for said potential source biomolecule;
calculating a weighted biomolecule score for said potential source biomolecule from said biomolecule score and said relative biomolecule match count; and
determining the likelihood of the presence or absence of said biomolecule based on said weighted biomolecule score.

12. The method of claim 1 wherein the step of determining the likelihood of the presence or absence of said biomolecule further comprises the steps of:
determining a biomolecule fragment count of said potential source biomolecule by counting the number of said mass signals that correspond to said potential source biomolecule; and
determining that said biomolecule is likely absent if said biomolecule fragment count of the corresponding potential source biomolecule is lower than a minimum number.

13. The method of claim 1 wherein the step of determining the likelihood of the presence or absence of said biomolecule further comprises the steps of:
determining a mass error for said mass signal from the relative difference between a mass which corresponds to said mass signal and a mass of a known biomolecule fragment which corresponds to said mass signal;
repeating the step of determining a mass error as necessary for additional mass signals that correspond to a known biomolecule fragment list for said potential source biomolecule;
selecting a mass tolerance value; and
determining that said biomolecule is likely absent if more than an insignificant number of said mass signals that correspond to said biomolecule have a said mass error that is greater than said selected mass tolerance value.

14. The method of claim 1 wherein the step of determining the likelihood of the presence or absence of said biomolecule further comprises the steps of:
identifying from about 100 to about 200 of the most intense mass signal intensities to determine the intense mass signals;
determining an intense biomolecule fragment count of said potential source biomolecule by counting the number of said intense mass signals that correspond to said potential source biomolecule; and
determining that said biomolecule is likely absent if said intense biomolecule fragment count of the corresponding potential source biomolecule is lower than a minimum number.

15. The method of claim 14 wherein the step of identifying comprises:
determining a mass error for said mass signal from the relative difference between a mass which corresponds to said mass signal and a mass of a known biomolecule fragment which corresponds to said mass signal;
selecting a mass tolerance value; and
identifying from about 100 to about 200 of the most intense mass signals with a said mass error less than said mass tolerance value and a biomolecule fragment score greater than a minimum number to determine the intense mass signals.

16. The method of claim 1 further comprising the step of determining a relative concentration of said biomolecule based on said biomolecule score.

17. The method of claim 1, further comprising the steps of:
combining at least two of said mass signal intensities to determine a signal intensity score;
combining said mass signal intensities of said mass signals which correspond to said known biomolecule fragment list for said potential source biomolecule to determine a potential source biomolecule intensity score;
comparing said signal intensity score to said potential source biomolecule intensity score to determine a relative biomolecule intensity corresponding to said potential source biomolecule; and
determining the likelihood of the presence or absence of said biomolecule based on said relative biomolecule intensity.

18. The method of claim 1, further comprising the steps of:
combining at least two of said biomolecule fragment scores to determine a signal biomolecule fragment score;
comparing said signal biomolecule fragment score to a said potential source biomolecule score to determine a relative biomolecule detection parameter corresponding to said potential source biomolecule; and
determining the likelihood of the presence or absence of said biomolecule based on said relative biomolecule detection parameter.

19. The method of claim 1, further comprising the steps of:
determining a mass error for said mass signal from the relative difference between a mass which corresponds to said mass signal and a mass of a known biomolecule fragment which corresponds to said mass signal;
repeating the step of determining a mass error as necessary for additional mass signals that correspond to a known biomolecule fragment list for said potential source biomolecule;
combining said mass errors for said mass signals corresponding to said potential source biomolecule to determine a biomolecule mass error for said potential source biomolecule;
repeating the steps determining a mass error and combining said mass errors as necessary for additional potential source biomolecules; and
determining the likelihood of the presence or absence of said biomolecule based on said biomolecule mass error.

20. The method of claim 19 wherein the step of combining said mass errors further comprises weighting a said mass error by the corresponding mass signal intensity to determine a weighted mass error and combining said weighted mass errors to determine said combined biomolecule mass error.

21. The method of claim 1 wherein said step of determining the likelihood of the presence or absence of said biomolecule comprises determining the likelihood of the presence or absence of a protein.

22. The method of claim 1 wherein said step of comparing said mass signals to a list of known biomolecule fragments signals comprises comparing said mass signals to a list of known peptides.

23. A method of determining the likelihood of the presence of a biomolecule comprising the steps of:
providing at least one mass signal, wherein said mass signal has a mass signal intensity;
comparing said mass signal to a list of known biomolecule fragment signals to determine a potential source biomolecule of said mass signal, wherein said mass signal corresponds to at least one biomolecule fragment of said potential source biomolecule;
determining a biomolecule fragment score for said mass signal, wherein said biomolecule fragment score comprises a function of a detection likelihood for said mass signal which defines a biomolecule fragment detection parameter;
repeating the steps of comparing and determining a biomolecule fragment score as necessary for additional mass signals;
combining said biomolecule fragment scores of said mass signals that correspond to a known biomolecule fragment list for said potential source biomolecule, said combination defining a biomolecule score for said potential source biomolecule;
repeating the step of combining as necessary for additional potential source biomolecules; and
determining whether said biomolecule should be subjected to MS-MS analysis based on said biomolecule score of said potential source biomolecule that corresponds to said biomolecule.

24. The method of claim 23 wherein said biomolecule fragment detection parameter is proportional to the detection efficiency of said biomolecule fragment that corresponds to said mass signal and a probability that said potential source biomolecule yields said biomolecule fragment as a result of a fragmentation process applied to said potential source biomolecule.

25. The method of claim 23 wherein the step of determining a biomolecule fragment score for said mass signal comprises the steps of:
determining a detection likelihood for said mass signal which defines said biomolecule fragment detection parameter;
determining a mass error for said mass signal from the relative difference between a mass which corresponds to said mass signal and a mass of a known biomolecule fragment which corresponds to said mass signal; and
determining said biomolecule fragment score from said mass signal intensity, said biomolecule fragment detection parameter, and said mass error for said mass signal.

26. The method of claim 25 wherein said biomolecule fragment score is proportional to said biomolecule fragment detection parameter and said mass signal intensity, and inversely proportional to said mass error for said mass signal.

27. The method of claim 23 wherein the step of combining said biomolecule fragment scores comprises excluding a selected biomolecule fragment score from said combination.

28. An article of manufacture having computer-readable program means for performing the method of claim 1 embodied thereon.

29. An article of manufacture having computer-readable program means for performing the method of claim 23 embodied thereon.

30. An apparatus for determining the likelihood of the presence of a biomolecule based on biomolecule fragment detection likelihood, the apparatus comprising:
a biomolecule fragment separation apparatus providing at least one mass signal, wherein said mass signal has a mass signal intensity;
a first memory element storing said mass signal provided by said biomolecule fragment separation apparatus;
a second memory element storing a list of known biomolecule fragment signals;
a third memory element storing a comparator accessing said first memory element and said second memory element to determine if a mass signal stored in said first memory element matches a biomolecule fragment signal in said second memory element;
a fourth memory element storing a mass signal-biomolecule fragment signal match determined by said comparator;
a fifth memory element containing a weight generator accessing said first memory element and said fourth memory element for determining a biomolecule fragment score for said mass signal;
a sixth memory element storing the biomolecule fragment scores;
a seventh memory element containing a combination generator accessing said third memory element and said sixth memory element for combining said biomolecule fragment scores of said mass signals that correspond to a known biomolecule fragment list for a potential source biomolecule, said combination defining a biomolecule score for said potential source biomolecule; and
an output device providing an output display of at least one of said biomolecule scores.

31. The apparatus of claim 30 wherein said biomolecule fragment separation apparatus comprises a MALDI ionization instrument.

32. The apparatus of claim 30 wherein said biomolecule fragment separation apparatus comprises a time-of-flight mass spectrometer.

33. The apparatus of claim 30 wherein said output device comprises a computer.
